(19) United States
(12) Patent Application Publication
(10) Pub. No.: US 2025/0008970 A1
(43) Pub. Date: Jan. 9, 2025

(54) MILK PRODUCT COMPOSITIONS

(71) Applicant: BIOMILQ, Inc., Durham, NC (US)

(72) Inventor: Leila Strickland, Hillsborough, NC (US)

(21) Appl. No.: 18/712,668

(22) PCT Filed: Nov. 23, 2022
(86) PCT No.: PCT/US2022/050920

§ 371 Date: May 22, 2024

Related U.S. Application Data
(60) Provisional application No. 63/264,501, filed on Nov. 23, 2021.

(51) Int. Cl. A23C 9/00; C12N 5/071

(52) U.S. Cl. CPC A23C 9/00; C12N 5/0631; C12N 2511/00; C12N 2513/00

(57) ABSTRACT

Disclosed herein are milk product compositions that comprise protein, lipid, and oligosaccharide components at concentrations that mimic and/or are substantially similar to cow milk. The milk product compositions include one or more milk components produced in vitro and/or ex vivo from cultured cow mammary cells.

### CROSS-REFERENCE

This application claims the benefit of and priority to U.S. Provisional Patent Application No. 63/264,501, filed Nov. 23, 2021, which is hereby incorporated by reference in its entirety for all purposes.

### BACKGROUND

Milk is a complex suspension of nutrients, including fats, sugars, proteins, vitamins, and minerals. Humans have consumed the milks of other species since prehistoric times, and dairy remains a staple of the human diet. Cow species are the most commonly used in dairy production, based on their high productivity and their widespread availability and adaptability as a species. However, dairy production is an agriculturally intensive process with substantial environmental impacts, including contributions to greenhouse gas production, as well as detrimental effects on land and water resources. Therefore, there is a need for improved milk products that reduce or eliminate the environmental impact of conventional, cow-based milk production.

### SUMMARY OF THE DISCLOSURE

Disclosed herein, in certain embodiments, are milk product compositions that comprise protein, lipid, and oligosaccharide components and component concentrations that mimic and/or are substantially similar to cow milk and are produced in vitro and/or ex vivo from cultured cow mammary cells.

In some embodiments of the milk product, the protein component comprises about 21-50 g/L of the milk product, and in some embodiments, the protein component can comprise one or more of whey protein and casein protein. Casein protein, in some embodiments, can comprise one or more of beta-casein, kappa-casein, and alpha-casein, and wherein, in some embodiments, the alpha-casein can comprise one or more of alpha_{S1}-casein and alpha_{S2}-casein.

In some embodiments, the protein component further comprises one or more of beta-lactoglobulin, alpha-lactalbumin, lysozyme, lactoferrin, and serum albumin.

In some embodiments of the milk product, the lipid component comprises about 35-55 g/L of the milk product, and in some embodiments of the milk product, the lipid component can comprise one or more of triacylglycerides, diacylglycerides, saturated fatty acids, monounsaturated fatty acids, polyunsaturated fatty acids, cholesterol, and phospholipids. In some embodiments, the saturated fatty acid component can comprise one or more of myristic acid, palmitic acid, and lauric acid. In some embodiments, palmitic acid is enriched in sn-2 configuration.

In some embodiments, the monounsaturated fatty acids comprise oleic acid.

In some embodiments, the polyunsaturated fats comprise one or more of linoleic acid, conjugated linoleic acid, and alpha-linoleic acid.

Some embodiments of the milk product comprise cholesterol.

Some embodiments of the milk product comprise one or more of phospholipids.

In some embodiments of the milk product, the milk oligosaccharide comprise about 0.01-0.15 g/L, and in some embodiments, the milk oligosaccharide component can comprise one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL).

Disclosed herein, in certain embodiments, the milk product comprises about 28-40 grams per liter (g/L) protein components, about 35-55 g/L lipid components, about 0.01-0.15 g/L milk oligosaccharides (MOs), and about 40-60 g/L lactose, wherein the protein components comprise one or more of whey, beta-casein, kappa-casein, alpha_{S1}-casein, alpha_{S2}-casein, beta-lactoglobulin, alpha-lactalbumin, lysozyme, lactoferrin, and serum albumin, wherein the lipid components comprise one or more of triacylglycerides, diacylglycerides, saturated fatty acids, monounsaturated fatty acids, polyunsaturated fatty acids, cholesterol, and phospholipids, wherein the milk oligosaccharide component comprises one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL), wherein saturated fatty acids comprise one or more of myristic acid, palmitic acid, and lauric acid, wherein monounsaturated fatty acids comprise oleic acid, wherein polyunsaturated fatty acids comprise one or more of linoleic acid, conjugated linoleic acid, and alpha-linoleic acid, and wherein at least one of the protein components, lipid components, MOs, and lactose is produced by cultured cow mammary epithelial cells.

Some embodiments of the milk product are isolated from cultured cow mammary epithelial cells (i.e., isolated from the secretion that is produced by cultured cow mammary epithelial cells and/or the cell culture supernatant), which, in some embodiments, comprise one or more immortalized cow mammary cell lines. In some embodiments, the cultured cow mammary epithelial cells are derived from one or more primary cow mammary tissue samples, which, in some embodiment, derive from needle aspiration, surgical explant of cow mammary gland tissue or other type of tissue removal method. In some embodiments, the cultured cow mammary epithelial cells are isolated from raw cow milk or descend from one or more cow mammary epithelial cells that were originally isolated from raw cow milk. In some embodiments, the one or more primary cow mammary tissue samples can comprise tissue or cells collected from cow mammary parenchyma.

In some embodiments, the primary cow mammary tissue further comprises one or more myoepithelial cells and/or comprises one or more stem cells.

In some embodiments, the milk product is sterile, and in some embodiments, the milk product is sterile without pasteurization. In some embodiments, the milk product is free of immunoglobulin protein. In some embodiments, the milk product comprises at least about 80% of the overall macromolecular composition of cow milk, or at least about 85% of the overall macromolecular composition of cow milk. In some embodiments, the milk product comprises at least about 90% of the overall macromolecular composition of cow milk. In some embodiments, the milk product comprises at least about 95% of the overall macromolecular composition of cow milk. In some embodiments, the milk product comprises at least about 97% of the overall macromolecular composition of cow milk. In some embodiments, the milk product comprises at least about 98% of the overall macromolecular composition of cow milk. In some embodiments, the milk product comprises at least about 99% of the overall macromolecular composition of cow milk. In some embodiments, non-protein nitrogen content comprises at least about 10% of total nitrogen content. In some embodiments, non-protein nitrogen content comprises at least about 15% of total nitrogen content. In some embodiments, non-protein nitrogen content comprises at least about 20% of total nitrogen content. In some embodiments, non-protein nitrogen content comprises at least about 25% of total nitrogen content. In some embodiments, non-protein nitrogen content comprises at least about 30% of total nitrogen content.

Disclosed herein, in certain embodiments, is a frozen milk product, a lyophilized milk product, a filtered milk product, an extracted milk product, and a containerized milk product comprising the milk product of the disclosure.

In some embodiments, the milk product can comprise between about 450-900 kcal/L available energy content, and in some embodiments, between about 40-60% of the available energy content is from lipid components in the milk product.

Disclosed herein, in certain embodiments, is a cow milk product, comprising: a. about 28-40 grams per liter (g/L) protein components; b. about 35-55 g/L lipid components; c. about 0.01-0.15 g/L milk oligosaccharides (MOs); and d. about 40-60 g/L lactose, wherein the protein components, lipid components, MOs, and lactose are produced by cultured cow mammary epithelial cells. In some embodiments, the protein component comprises whey protein. In some embodiments, the whey protein has a concentration of about 1-24 g/L in the milk product. In some embodiments, the protein component further comprises casein protein. In some embodiments, the protein component comprises casein protein. In some embodiments, the protein component comprises beta-casein, kappa-casein and alpha-casein. In some embodiments, the beta-casein has a concentration of about 7-12 g/L, the kappa-casein has a concentration of about 1-4 g/L, and the alpha-casein has a concentration of about 9-16 g/L in the milk product. In some embodiments, the alpha-casein comprises one or more of alphaS1-casein and alphaS2-casein. In some embodiments, the alphaS1-casein is more than about 3-fold more abundant than alphaS2-casein. In some embodiments, the alphaS1-casein has a concentration of about 7-12 g/L in the milk product. In some embodiments, the alphaS2-casein has a concentration of about 2-4 g/L in the milk product. In some embodiments, the beta-casein comprises greater than about 50% of total casein content. In some embodiments, the protein component further comprises one or more of beta-lactoglobulin, alpha-lactalbumin, lysozyme, lactoferrin and serum albumin. In some embodiments, the beta-lactoglobulin has a concentration of about 2-5 g/L in the milk product. In some embodiments, the milk product does not compromise beta-lactoglobulin. In some embodiments, the alpha-lactalbumin has a concentration of about 0.5-2 g/L in the milk product. In some embodiments, the lysozyme has a concentration of about 5-15 μg/L in the milk product. In some embodiments, the lactoferrin has a concentration of about 0.01-0.5 g/L in the milk product. In some embodiments, the serum albumin has a concentration of about 0.05-2 g/L in the milk product. In some embodiments, the lipid component comprises one or more of triacylglycerides, diacylglycerides, saturated fatty acids, monounsaturated fatty acids, polyunsaturated fatty acids, cholesterol, and phospholipids. In some embodiments, the triacylglycerides have a concentration of about 30-54 g/L in the milk product. In some embodiments, the diacylglycerides have a concentration of about 0.33-2 g/L in the milk product. In some embodiments, the saturated fatty acids have a concentration of about 15-25 g/L in the milk product. In some embodiments, the saturated fatty acids comprise one or more of myristic acid, palmitic acid, and lauric acid. In some embodiments, myristic acid has a concentration of about 1-4 g/L in the milk product. In some embodiments, palmitic acid has a concentration of about 6-10 g/L in the milk product. In some embodiments, lauric acid has a concentration of about 0.6-1 g/L in the milk product. In some embodiments, monounsaturated fatty acids have a concentration of about 5-12 g/L in the milk product. In some embodiments, the monounsaturated fatty acid comprises oleic acid. In some embodiments, the oleic acid has a concentration of about 6-10 g/L in the milk product. In some embodiments, polyunsaturated fats have a concentration of about 0.5-10 g/L in the milk product. In some embodiments, the polyunsaturated fats comprise one or more of linoleic acid, conjugated linoleic acid, and alpha-linoleic acid. In some embodiments, linoleic acid has a concentration of about 0.5-2 g/L in the milk product. In some embodiments, conjugated linolenic acid has a concentration of about 0.05-0.15 g/L in the milk product. In some embodiments, alpha-linoleic acid has a concentration of about 0.5-1.5 g/L in the milk product. In some embodiments, linoleic acid has a concentration of about 0.5-2 g/L, conjugated linolenic acid has a concentration of about 0.05-0.15 g/L, and alpha-linoleic acid has a concentration of about 0.5-1.5 g/L in the milk product. In some embodiments, cholesterol has a concentration of about 0.2-4 g/L in milk product. In some embodiments, phospholipids have a concentration of about 0.1-1 g/L in the milk product. In some embodiments, the phospholipids have a concentration of about 10-45 g/L in the milk product. In some embodiments, the milk oligosaccharide component comprises one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL). In some embodiments, the one or more milk oligosaccharides comprises 6′-Sialyllactose (6′-SL), which oligosaccharide has a concentration of about 0.01-0.1 g/L in the milk product. In some embodiments, the one or more oligosaccharides comprises 6′-sialyl-n-acetyllactosamine (6′-SLN), which oligosaccharide has a concentration of about 0.005-0.02 g/L in the milk product. In some embodiments, the one or more oligosaccharides comprises Disialyllactose (DSL), which oligosaccharide has a concentration of less than about 0.01 g/L in the milk product. In some embodiments, the one or more oligosaccharides comprises Galactosaminuyllactose (GNL), which oligosaccharide has a concentration of about 0.002-0.006 g/L in the milk product. In some embodiments, the one or more oligosaccharides comprises 3′-Sialyllactose (3′-SL), which oligosaccharide has a concentration of about 0.025-0.15 g/L in the milk product. In some embodiments, the milk product comprises about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL), and about 0.025-0.15 g/L 3′-SL (3′-sialyllactose).

Disclosed herein, in some embodiments, is a cow milk product comprising: a. about 28-40 grams per liter (g/L) protein components; b. about 35-55 g/L lipid components; c. about 0.01-0.15 g/L milk oligosaccharides (MOs); and d. about 40-60 g/L lactose, wherein the protein components comprise whey, beta-casein, kappa-casein, alphaS1-casein, alphaS2-casein, alpha-lactalbumin, lysozyme, lactoferrin, and serum albumin, wherein the lipid components comprise triacylglycerides, diacylglycerides, saturated fatty acids, monounsaturated fatty acids, polyunsaturated fatty acids, cholesterol, and phospholipids, wherein the milk oligosaccharides comprise 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL), wherein saturated fatty acids comprise myristic acid, palmitic acid, and lauric acid, wherein monounsaturated fatty acids comprises oleic acid, wherein polyunsaturated fatty acids comprise linoleic acid, conjugated linoleic acid, and alpha-linoleic acid, and wherein the protein components, lipid components, MOs, and lactose are produced by cultured cow mammary epithelial cells. In some embodiments, the milk product comprises about 2-16 g/L whey, 7-12 g/L beta-casein, about 1-4 g/L kappa-casein, about 7-12 g/L alphaS1-casein, about 2-4 g/L alphaS2-casein, about 2-5 g/L beta-lactoglobulin, about 0.5-2 g/L alpha-lactalbumin, about 5-15 μg/L lysozyme, about 0.01-0.5 g/L lactoferrin, about 0.05-2 g/L serum albumin, about 30-54 g/L triacylglycerides, about 0.3-2 g/L diacylglycerides, about 15-25 g/L saturated fatty acids, about 5-12 g/L monounsaturated fatty acids, about 0.5-10 g/L polyunsaturated fatty acids, about 0.2-4 g/L cholesterol, about 0.1-1 g/L phospholipids, about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL) and about 0.025-0.15 g/L 3′-Sialyllactose (3′-SL), wherein the saturated fatty acids comprise about 1-4 g/L myristic acid, about 6-10 g/L palmitic acid, and about 0.6-1 g/L lauric acid, wherein the monounsaturated fatty acids comprises about 6-10 g/L oleic acid, and wherein the polyunsaturated fatty acids comprise about 0.5-2 g/L linoleic acid, about 0.05-0.15 g/L conjugated linoleic acid, and about 0.5-1.5 g/L alpha-linoleic acid. In some embodiments, the protein components, lipid components, MOs, and lactose are isolated from cultured cow mammary epithelial cells. In some embodiments, the cultured cow mammary epithelial cells comprise one or more immortalized cow mammary cell lines. In some embodiments, the cultured cow mammary epithelial cells are derived from one or more primary cow mammary tissue samples. In some embodiments, the one or more primary cow mammary tissue samples is derived from a surgical explant of cow mammary gland tissue. In some embodiments, the one or more primary cow mammary tissue samples comprises tissue or cells collected from cow mammary parenchyma. In some embodiments, the one or more primary cow mammary tissue samples is derived from a needle aspiration of cow mammary gland tissue. In some embodiments, the primary cow mammary tissue further comprises one or more myoepithelial cells. In some embodiments, the primary cow mammary tissue further comprises one or more stem cells. In some embodiments, the cultured cow mammary epithelial cells are co-cultured with one or more myoepithelial cell lines. In some embodiments, the cultured cow mammary epithelial cells are co-cultured with one or more stem cell lines.

Disclosed herein, in certain embodiments, is a milk product that is sterile. In some embodiments, the milk product is sterile without pasteurization. In some embodiments, the milk product is free of immunoglobulin protein. In some embodiments, the milk product comprises at least about 80%, or at least about 85%, or at least about 90%, or at least about 95%, or at least about 97%, or at least about 98%, or at least about 99%, of the overall macromolecular composition of cow milk. In some embodiments, non-protein nitrogen content comprises at least about 10%, or at least about 15%, or at least about 20%, or at least about 25%, or at least about 30% of total nitrogen content.

Disclosed herein, in certain embodiments, is a frozen milk product, comprising the milk product that has been frozen.

Disclosed herein, in certain embodiments, is a lyophilized milk product, comprising the milk product that has been lyophilized.

Disclosed herein, in certain embodiments, is a containerized milk product, comprising the milk product, that is packaged into a container.

Disclosed herein, in certain embodiments, is a containerized frozen milk product that is packaged into a container.

Disclosed herein, in certain embodiments, is a containerized lyophilized milk product, comprising the lyophilized milk product that is packaged into a container.

Disclosed herein, in certain embodiments, is an extracted milk product, comprising one or more components extracted from the milk product. In some embodiments, the one or more components extracted from the collected milk product are lyophilized or concentrated to produce a lyophilized or a concentrated extracted milk product component. In some embodiments, the one or more components extracted from the collected milk product are concentrated by membrane filtration or reverse osmosis. In some embodiments, the one or more extracted components from the collected milk product comprise milk protein, lipid, carbohydrate, vitamin, and minerals.

Disclosed herein, in certain embodiments, is containerized extracted milk product that is packaged in a container. In some embodiments, the container is sterile. In some embodiments, the container is vacuum-sealed. In some embodiments, the container is a food grade container. In some embodiments, the container is a canister, ajar, a bottle, a bag, a box, or a pouch.

In some embodiments, the milk product comprises about 450-900 kcal/L available energy content. In some embodiments, between about 40-60% of the available energy content is from lipids. In some embodiments, the milk product comprises between about 100 and 160 g/L macromolecular content. In some embodiments, the casein protein is complexed into one or more micelle structures with a diameter between about 100 nm and 200 nm. In some embodiments, the milk product comprises between about 109 to 1011 milk fat globules per milliliter, wherein the milk fat globules have a core comprising one or more triacylglycerides, the core surrounded by a trilayer comprising one or more phospholipids and one or more membrane proteins. In some embodiments, between about 30%-50% of the triacylglycerides present in the milk product are substituted with palmitic acid (C16:0) at a sn-2 position. In some embodiments, the milk product comprises an omega-6 to omega-3 fatty acid ratio of about 1.5 to 4.5. In some embodiments, the milk product comprises the milk product comprises between about 100-160 g/L macromolecular content.

Disclosed herein, in certain embodiments, is filtered milk product, comprising one or more components filtered from the milk product. In some embodiments, the one or more components filtered from the collected milk product are lyophilized or concentrated to produce a lyophilized or a concentrated filtered milk product component. In some embodiments, the one or more components filtered from the collected milk product are concentrated by membrane filtration or reverse osmosis. In some embodiments, the one or more filtered components from the collected milk product comprise milk protein, lipid, carbohydrate, vitamin, and minerals. In some embodiments, the protein components, lipid components, MOs, and lactose are isolated from a secretion that is produced by the cultured cow mammary epithelial cells. In some embodiments, the protein components, lipid components, MOs, and lactose are isolated from a supernatant from the cultured cow mammary epithelial cells. In some embodiments, the cultured cow mammary epithelial cells are derived from one or more cow mammary epithelial cells isolated from raw cow milk. In some embodiments, the antioxidant capacity of the milk products is substantially different from the antioxidant capacity of cow milk. In some embodiments, the milk product does not comprise or is substantially free of one or more contaminants and toxins of cow milk. In some embodiments, the milk product does not comprise or is substantially free of one or more contaminants of cow milk selected from the group comprising hormones (e.g., pituitary, steroid, hypothalamic, and thyroid hormones), gastrointestinal peptides (e.g., nerve and epidermal growth factors, and the growth inhibitors MDGI and MAF), rBGH or recombinant cow growth hormone (a genetically engineered hormone injected into cows to increase milk production), pus from infected cow udders, and/or antibiotics or pharmaceuticals which have been administered to cows. In some embodiments, the milk product does not comprise or is substantially free of one or more pathogens or microorganisms of cow milk. In some embodiments, the milk product does not comprise or is substantially free of one or more pathogens or microorganisms of cow milk selected from the group comprising Brucella, Campylobacter jejuni, Coliforms, Coxiella burnetii, Escherichia coli, Listeria monocytogenes, Mycobacterium bovis and tuberculosis, Mycobacterium paratuberculosis, Psychrotrophic Bacteria, Salmonella spp., Yersinia enterocolitica, Proteobacteria, Bacteroidetes, Firmicutes, Actinobacteria, Acidobacteria, Pseudomonas, Brevibacteriaceae, Corynebacteriaceae, Staphylococcaceae, Arthrobacter, Cronobacter, Ruminococcus and/or Faecalibacterium.

Disclosed herein, in certain embodiments, is a method of producing an isolated milk product from non-human mammary cells, the method comprising: a. culturing a cell construct in a bioreactor under conditions which produce the milk product, said cell construct comprising: i. a three-dimensional scaffold having an exterior surface, an interior surface defining an interior cavity, and a plurality of pores extending from the interior surface to the exterior surface; ii. a matrix material disposed on the exterior surface of the three-dimensional scaffold; iii. a culture media disposed within the interior cavity and in fluidic contact with the internal surface; iv. a plurality of plasma cells disposed on the matrix material; and v. a confluent monolayer of polarized non-human mammary cells disposed on the plurality of plasma cells, wherein the mammary cells are selected from the group consisting of: mammary epithelial cells, mammary myoepithelial cells, and mammary progenitor cells, wherein the polarized mammary cells comprise an apical surface and a basal surface; and b. isolating the cultured milk product. In some embodiments, the non-human mammary cells are cow mammary cells, bison mammary cells, buffalo mammary cells, yak mammary cells, goat mammary cells, sheep mammary cells, reindeer mammary cells, camel mammary cells, pig mammary cells, dog mammary cells, cat mammary cells, and equine mammary cells. In some embodiments, the plasma cells are non-human plasma cells. In some embodiments, the non-human plasma cells are cow plasma cells, bison plasma cells, buffalo plasma cells, yak plasma cells, goat plasma cells, sheep plasma cells, reindeer plasma cells, camel plasma cells, pig plasma cells, dog plasma cells, cat plasma cells, and equine plasma cells. In some embodiments, the plasma cells are human plasma cells. In some embodiments, the monolayer of polarized non-human mammary cells is at least 70% confluent, at least 80% confluent, at least 90% confluent, at least 95% confluent, at least 99% confluent, or 100% confluent. In some embodiments, at least 70%, at least 80%, at least 90%, at least 95%, at least 99%, or 100% of the non-human mammary cells are polarized in the same orientation. In some embodiments, the milk product comprises secretory IgA (sIgA). In some embodiments, the bioreactor comprises an apical compartment that is substantially isolated from the internal cavity of the cell construct. In some embodiments, the basal surface of the non-human mammary cells is in fluidic contact with the culture media. In some embodiments, the apical compartment is in fluidic contact with the apical surface of the non-human mammary cells. In some embodiments, the milk product is secreted from the apical surface of the non-human mammary cells into the apical compartment. In some embodiments, the culture media substantially does not contact the milk product. In some embodiments, total cell density of non-human mammary cells within the bioreactor is at least 10¹¹; and alternatively wherein total surface area of mammary cells within the bioreactor is at least 1.5 m². In some embodiments, total cell density of plasma cells in the bioreactor is about 200 to 500 plasma cells per mm². In some embodiments, the culturing is carried out at a temperature of about 27° C. to about 39° C. In some embodiments, the culturing is carried out at an atmospheric concentration of CO2 of about 4% to about 6%.

Disclosed herein, in certain embodiments, is a cell construct, comprising: a. a three dimensional scaffold having an exterior surface, an interior surface defining an interior cavity/basal chamber, and a plurality of pores extending from the interior surface to the exterior surface; b. a matrix material disposed on the exterior surface of the three-dimensional scaffold; c. a culture media disposed within the interior cavity/basal chamber and in fluidic contact with the internal surface; d. a plurality of plasma cells disposed on the matrix material; and e. an at least 70% confluent monolayer of polarized non-human mammary cells disposed on the plurality of plasma cells, wherein the non-human mammary cells are selected from the group consisting of: non-human mammary epithelial cells, non-human mammary myoepithelial cells, and non-human mammary progenitor cells. In some embodiments, the polarized non-human mammary cells comprise an apical surface and a basal surface. In some embodiments, the basal surface of the non-human mammary cells is in fluidic contact with the culture media. In some embodiments, at least 70%, at least 80%, at least 90%, at least 95%, at least 99%, or 100% of the non-human mammary cells are polarized in the same orientation. In some embodiments, the monolayer of polarized non-human mammary cells is at least 70% confluent, at least 80% confluent, at least 90% confluent, at least 95% confluent, at least 99% confluent, or 100% confluent. In some embodiments, the non-human mammary cells comprise a constitutively active prolactin receptor protein. In some embodiments, the culture medium comprises prolactin.

Disclosed herein, in certain embodiments, is a non-human milk product, comprising: a plurality of non-human milk proteins and/or lipids and/or oligosaccharides, wherein the milk product does not comprise or is substantially free of persistent organic pollutants (POPs), heavy metals, non-milk allergens, cells, hormones, or virus; provided that the cultured milk product may comprise a non-human mammary epithelial cell or a plasma cell (PC). In some embodiments, the milk product is a cow milk product, bison mil product, buffalo milk product, yak milk product, goat milk product, sheep milk product, camel milk product, reindeer milk product, pig milk product, dog milk product, cat milk product, or horse milk product. In some embodiments, the milk product is a cow milk product and does not comprise beta-lactoglobulin. In some embodiments, the milk product further comprises secretory IgA (sIgA). In some embodiments, the sIgA is human sIgA, cow sIgA, bison mil product, buffalo sIgA, yak sIgA, goat sIgA, sheep sIgA, camel sIgA, reindeer sIgA, pig sIgA, dog sIgA, cat sIgA, or horse sIgA. In some embodiments, the non-human milk product does not comprise or is substantially free of persistent organic pollutants (POPs). In some embodiments, the non-human milk product does not comprise or is substantially free of polychlorinated dibenzo-p-dioxins (PCDDs), polychlorinated dibenzofurans (PCDFs), polychlorinated biphenyls (PCBs) and pesticides. In some embodiments, the non-human milk product does not comprise or is substantially free of DDT. In some embodiments, the non-human milk product does not comprise or is substantially free of heavy metals. In some embodiments, the non-human milk product does not comprise or is substantially free of mercury, lead, arsenic, cadmium, nickel, chromium, cobalt, or zinc. In some embodiments, the non-human milk product does not comprise or is substantially free of non-milk allergens. In some embodiments, the non-human milk product does not comprise or is substantially free of ovomucoid, ovalbumin, conalbumin, arachin 6, arachin 3, conarachin, Arah1, and arachin Arah2. In some embodiments, the non-human milk product does not comprise or is substantially free of human stem cells, human immune cells, or bacterial cells, provided that the non-human milk product comprises one or more non-human mammary epithelial cells or plasma cells (PCs). In some embodiments, the non-human milk product does not comprise or is substantially free of myoepithelial cells, myeloid precursor cells, neutrophils, granulocytes, or T cells. In some embodiments, the non-human milk product does not comprise or is substantially free of Staphylococcus, Acinetobacter, Streptococcus, Pseudomonas, Lactococcus, Enterococcus or Lactobacillus. In some embodiments, the non-human milk product does not comprise or is substantially free of virus. In some embodiments, the non-human milk product does not comprise or is substantially free of hormones. In some embodiments, the non-human milk product does not comprise or is substantially free of leptin, ghrelin, adiponectin, thyroxine (T4), triiodothyronine (T3) thyroid-stimulating hormone (TSH), epidermal growth factor, beta-endorphin, relaxin, cortisol, or erythropoietin.

Disclosed herein, in certain embodiments, is a method of feeding a subject in need thereof, comprising administering to the subject a cultured milk product. In some embodiments, the subject is a human, for example a human infant or an immunocompromised human. In some embodiments, the human subject has a disease selected from: severe combined immunodeficiency (SCID), HIV/AIDS, a cancer, or an autoimmune disease. In some embodiments, the subject has lupus or diabetes (for example, Type I diabetes or Type II diabetes). In some embodiments, the human subject is an organ or bone marrow transplant recipient. In some embodiments, the human subject is malnourished. In some embodiments, the human subject has malabsorption syndrome. In some embodiments, the human subject has wasting syndrome. In some embodiments, the human subject is geriatric. In some embodiments, the subject is a cow, buffalo, bison, yak, goat, sheep, camel, pig, dog, cat or horse.

Disclosed herein, in certain embodiments, is method of treating or preventing a microbial infection in a subject in need thereof, comprising administering to the subject a cultured milk product. In some embodiments, the sIgA is human sIgA and subject is a human. In some embodiments, the sIgA is cow sIgA and subject is a cow. In some embodiments, the sIgA is buffalo sIgA and subject is a buffalo. In some embodiments, the sIgA is bison sIgA and subject is a bison. In some embodiments, the sIgA is yak sIgA and subject is a yak. In some embodiments, the sIgA is reindeer sIgA and subject is a reindeer. In some embodiments, the sIgA is camel sIgA and subject is a camel. In some embodiments, the sIgA is goat sIgA and subject is a goat. In some embodiments, the sIgA is sheep sIgA and subject is a sheep. In some embodiments, the sIgA is dog sIgA and subject is a dog. In some embodiments, the sIgA is cat sIgA and subject is a cat. In some embodiments, the sIgA is horse sIgA and subject is a horse. In some embodiments, the infection is a bacterial infection or a viral infection. In some embodiments, the microbial infection is a gastrointestinal infection. In some embodiments, the microbial infection is a respiratory infection.

Disclosed herein, in certain embodiments, is method of producing isolated non-human sIgA from non-human mammary cells, the method comprising: a. culturing a cell construct in a bioreactor under conditions which produce a non-human milk product, said cell construct comprising: i. a three-dimensional scaffold having an exterior surface, an interior surface defining an interior cavity, and a plurality of pores extending from the interior surface to the exterior surface; ii. a matrix material disposed on the exterior surface of the three-dimensional scaffold; iii. a culture media disposed within the interior cavity and in fluidic contact with the internal surface; iv. a plurality of non-human plasma cells disposed on the matrix material; and v. a confluent monolayer of polarized non-human mammary cells disposed on the plurality of non-human plasma cells, wherein the non-human mammary cells are selected from the group consisting of: non-human mammary epithelial cells, non-human mammary myoepithelial cells, and non-human mammary progenitor cells, wherein the polarized non-human mammary cells comprise an apical surface and a basal surface; and b. isolating the sIgA from the milk product. In some embodiments, the monolayer of polarized non-human mammary cells is at least 70% confluent, at least 80% confluent, at least 90% confluent, at least 95% confluent, at least 99% confluent, or 100% confluent. In some embodiments, at least 70%, at least 80%, at least 90%, at least 95%, at least 99%, or 100% of the non-human mammary cells are polarized in the same orientation. In some embodiments, the non-human milk product comprises non-human secretory IgA (sIgA). In some embodiments, the bioreactor comprises an apical compartment that is substantially isolated from the internal cavity of the cell construct. In some embodiments, the basal surface of the mammary cells is in fluidic contact with the culture media. In some embodiments, the apical compartment is in fluidic contact with the apical surface of the mammary cells. In some embodiments, the cultured milk product is secreted from the apical surface of the mammary cells into the apical compartment. In some embodiments, the culture media substantially does not contact the cultured milk product. In some embodiments, total cell density of mammary cells within the bioreactor is at least 10¹¹; and alternatively wherein total surface area of mammary cells within the bioreactor is at least 1.5 m². In some embodiments, total cell density of plasma cells in the bioreactor is about 200 to 500 plasma cells per mm². In some embodiments, the culturing is carried out at a temperature of about 27° C. to about 39° C. In some embodiments, the culturing is carried out at an atmospheric concentration of CO2 of about 4% to about 6%.

Disclosed herein, in certain embodiments, is method of treating or preventing a microbial infection in a non-human subject in need thereof, comprising administering to the non-human subject a immunotherapeutic composition comprising (a) non-human sIgA and (b) a pharmaceutically acceptable excipient, wherein the non-human sIgA is manufactured by a method disclosed herein. In some embodiments, the sIgA is human sIgA and subject is a human. In some embodiments, the sIgA is cow sIgA and subject is a cow. In some embodiments, the sIgA is buffalo sIgA and subject is a buffalo. In some embodiments, the sIgA is bison sIgA and subject is a bison. In some embodiments, the sIgA is yak sIgA and subject is a yak. In some embodiments, the sIgA is reindeer sIgA and subject is a reindeer. In some embodiments, the sIgA is camel sIgA and subject is a camel. In some embodiments, the sIgA is goat sIgA and subject is a goat. In some embodiments, the sIgA is sheep sIgA and subject is a sheep. In some embodiments, the sIgA is dog sIgA and subject is a dog. In some embodiments, the sIgA is cat sIgA and subject is a cat. In some embodiments, the sIgA is horse sIgA and subject is a horse. In some embodiments, the infection is a bacterial infection or a viral infection. In some embodiments, the microbial infection is a gastrointestinal infection. In some embodiments, the microbial infection is a respiratory infection. In some embodiments, the composition is formulated for inhalation. In some embodiments, the composition is a powder. In some embodiments, the composition is formulation for administration by a nebulizer.

Disclosed herein, in certain embodiments, is a pharmaceutical composition, comprising: sIgA manufactured by a method disclosed herein; and a pharmaceutically acceptable excipient. In some embodiments, the pharmaceutically acceptable excipient is a stabilizer, a surfactant, a buffer or tonicity agent. In some embodiments, the pharmaceutically acceptable excipient is sucrose, trehalose, mannitol, sorbitol, histidine, arginine, glycine, polysorbate 20, polysorbate 80, poloxamer 188, edetic acid/or edetate salts (e.g., EDTA), glutathione, metacresol, phenol, benzyl alcohol, benzalkonium chloride, methionine or cysteine.

Having described the present disclosure, the same will be explained in greater detail in the following examples, which are included herein for illustration purposes only, and which are not intended to be limiting to the disclosure.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 depicts a comparison of lipid profiles between naturally produced and cell cultured bovine milk product examples. Phospholipids comprise nearly half of the lipid content of two examples of cell cultured bovine milk products, whereas triglycerides predominate in naturally occurring bovine milk.

FIG. 2 depicts a comparison of phospholipid profiles between naturally produced and cell cultured bovine milk. Although phospholipids comprise a smaller proportion of the total lipid profile in cell cultured bovine milk compared with naturally produced bovine milk, the relative distribution of lipids across the major subclasses is similar.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiments, and features illustrated with respect to a particular embodiment may be deleted from that embodiment. Numerous variations and additions to the various embodiments suggested herein will be apparent to those skilled in the art in light of the instant disclosure, which do not depart from the instant disclosure. Hence, the following specification is intended to illustrate some particular embodiments of the disclosure, and not to exhaustively specify all permutations, combinations, and variations thereof.

Unless the context indicates otherwise, it is specifically intended that the various features described herein can be used in any combination. Moreover, in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this disclosure belongs. The terminology used in the description herein is for the purpose of describing particular embodiments only and is not intended to be limiting.

All publications, patent applications, patents, nucleotide sequences, amino acid sequences and other references mentioned herein are incorporated by reference in their entireties for all purposes.

### Definitions

The terminology used herein is for the purpose of describing particular embodiments only and is not intended to be limiting.

As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Furthermore, the term “about,” as used herein when referring to a measurable value such as an amount of a compound or agent of this disclosure, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the disclosure. Thus, the term “consisting essentially of” as used herein should not be interpreted as equivalent to “comprising.”

As used herein, the term “polypeptide” encompasses both peptides and proteins, and does not require any particular amino acid length or tertiary structure unless indicated otherwise.

The term “polarized” as used herein in reference to cells and/or monolayers of cells refers to a spatial status of the cell wherein there are two distinct surfaces of the cell, e.g., an apical surface and a basal surface, which may be different. The distinct surfaces of a polarized cell have different surface and/or transmembrane receptors and/or other structures.

As used herein, “apical surface” means the surface of a cell that faces an external environment or toward a cavity, for example the cavity of an internal organ. With respect to mammary epithelial cells, the apical surface is the surface from which the milk product is excreted.

As used herein, “basal surface” means the surface of a cell that is in contact with a surface, e.g., the extracellular matrix of a bioreactor.

As used herein, “bioreactor” means a device or system that supports a biologically active environment that enables the production of a culture milk product described herein from mammary cells described herein.

The term “lactogenic” as used herein refers to the ability to stimulate production and/or secretion of milk. A gene or protein (e.g., prolactin) may be lactogenic, as may any other natural and/or synthetic product. In some embodiments, a lactogenic culture medium comprises prolactin, thereby stimulating production of milk by cells in contact with the culture medium.

### Cell Constructs

Disclosed herein are cell constructs for producing a non-human mammal (e.g., cow, bison, buffalo, yak, goat, sheep, camel, reindeer, pig, dog, cat, or horse) milk product representing the biosynthetic output of non-human (e.g., cow, bison, buffalo, yak, goat, sheep, camel, reindeer, pig, dog, cat, or horse) mammary epithelial cells (immortalized or from primary tissue samples) and IgA producing cells, for example plasma cells. Disclosed herein, in certain embodiments, are cell constructs for producing non-human (e.g., cow, bison, buffalo, yak, goat, sheep, camel, reindeer, pig, dog, cat, or horse) milk products comprising sIgA, the cell constructs comprising: (a) a three dimensional scaffold having an exterior surface, an interior surface defining an interior cavity/basal chamber, and a plurality of pores extending from the interior surface to the exterior surface; (b) a matrix material disposed on the exterior surface of the three-dimensional scaffold; (c) a culture media disposed within the interior cavity/basal chamber and in fluidic contact with the internal surface; (d) a population of plasma cells (PCs) disposed on the matrix material, and (e) a continuous monolayer of non-human (e.g., cow, bison, buffalo, yak, goat, sheep, camel, reindeer, pig, dog, cat, or horse) mammary cells disposed on the population of plasma cells, the non-human mammary cells selected from the group consisting of: (i) non-human mammary epithelial cells, (ii) non-human mammary myoepithelial cells, and (iii) non-human mammary progenitor cells.

### Non-Human Mammary Cells

In some embodiments, the non-human mammary cells are cattle mammary cells, bison mammary cells, buffalo mammary cells, yak mammary cells, horse mammary cells, goat mammary cells, sheep mammary cells, camel mammary cells, reindeer mammary cells, pig mammary cells, cat mammary cells, or dog mammary cells. In some embodiments, the non-human mammary cells comprise milk-producing non-human mammary epithelial cells (MECs), non-human contractile myoepithelial cells, and/or non-human progenitor cells that can give rise to both non-human mammary epithelial cells (MECs) and non-human mammary contractile myoepithelial cells. In some embodiments, the non-human mammary cells comprise non-human mammary epithelial cells (MECs), primary non-human mammary epithelial cells, non-human mammary myoepithelial cells and non-human mammary progenitor cells.

In some embodiments, at least 50% of the non-human mammary cells of the cells culture are polarized. In some embodiments, at least 55% of the non-human mammary cells of the cell culture are polarized. In some embodiments, at least 60% of the non-human mammary cells of the cell culture are polarized. In some embodiments, at least 65% of the non-human mammary cells of the cell culture are polarized. In some embodiments, at least 70% of the non-human mammary cells of the cell culture are polarized. In some embodiments, at least 75% of the non-human mammary cells of the cell culture are polarized. In some embodiments, at least 80% of the non-human mammary cells of the cell culture are polarized. In some embodiments, at least 85% of the non-human mammary cells of the cell culture are polarized. In some embodiments, at least 90% of the non-human mammary cells of the cell culture are polarized. In some embodiments, at least 95% of the non-human mammary cells of the cell culture are polarized. In some embodiments, at least 100% of the non-human mammary cells of the cell culture are polarized. In some embodiments, substantially all of the non-human mammary cells of the cell construct are polarized (i.e., have an apical surface and a basal surface). In some embodiments, substantially all the non-human mammary cells of the cell construct are polarized and substantially all the polarized cells are oriented in the same direction. For example, in some embodiments, substantially all of the non-human mammary cells have an apical surface and a basal surface, wherein the apical surface of substantially all of the cells is oriented in the same direction and the basal surface of substantially all of the cells is oriented in the same direction.

In some embodiments, the continuous monolayer of non-human mammary cells has at least 70% confluence over the scaffold. In some embodiments, the continuous monolayer of non-human mammary cells has at least about 75% confluence over the scaffold. In some embodiments, the continuous monolayer of non-human mammary cells has at least about 80% confluence over the scaffold. In some embodiments, the continuous monolayer of non-human mammary cells has at least about 85% confluence over the scaffold. In some embodiments, the continuous monolayer of non-human mammary cells has at least about 90% confluence over the scaffold. In some embodiments, the continuous monolayer of non-human mammary cells has at least about 95% confluence over the scaffold. In some embodiments, the continuous monolayer of non-human mammary cells has at least about 99% confluence over the scaffold. In some embodiments, the continuous monolayer of non-human mammary cells has 100% confluence over the scaffold.

### Genetic Modifications to Mammary Cells

In some embodiments, the non-human mammary cells comprise a constitutively active prolactin receptor protein. In some embodiments, the non-human mammary cells comprise a constitutively active non-human mammal prolactin receptor protein. Where the non-human mammal primary mammary epithelial cell or the immortalized non-human mammary epithelial cells comprise a constitutively active prolactin receptor, the culture medium does not contain prolactin.

In some embodiments, the non-human mammary cells are modified, where applicable, to not comprise or not express a beta-lactoglobulin gene (Blg). Beta-lactoglobulin is a primary human allergen in the milk of certain non-human mammals, for example cows, sheep, goat, yak, buffalo, bison, and reindeer. It is the main whey protein, without any counterpart in human milk. Any suitable method is used to excise or inactive the Blg gene.

In some embodiments, cow mammary cells are modified to not comprise or express a beta-lactoglobulin gene (Blg). In some embodiments, yak mammary cells are modified to not comprise or express a beta-lactoglobulin gene (Blg). In some embodiments, goat mammary cells are modified to not comprise or express a beta-lactoglobulin gene (Blg). In some embodiments, buffalo mammary cells are modified to not comprise or express a beta-lactoglobulin gene (Blg). In some embodiments, bison mammary cells are modified to not comprise or express a beta-lactoglobulin gene (Blg). In some embodiments, sheep mammary cells are modified to not comprise or express a beta-lactoglobulin gene (Blg). In some embodiments, reindeer mammary cells are modified to not comprise or express a beta-lactoglobulin gene (Blg).

### Plasma Cells

Plasma cells produce one or more immunoglobins of a class selected from IgG, IgM and IgA. In certain instances, IgA produced by plasma cells is processed by mammary epithelial cells to yield sIgA. sIgA comprises a secretory component, the extracellular domain of the polymeric Ig receptor, attached to an IgA. Mammary epithelial cells process IgA by cleaving the extracellular domain of a polymeric Ig receptor to generate sIgA. In certain instances, the sIgA is secreted by the apical surface of the mammary cells in to a milk product, for example a milk product described herein.

In certain embodiments, the plasma cells are cultivated with the non-human mammary epithelial cells on a scaffold, thereby producing a cell construct for producing a milk product with secretory products of the plasma cells and non-human mammary cells (e.g., sIgA). In certain embodiments, the plasma cells are grown on a scaffold below a monolayer of non-human mammary cells. In certain embodiments, the plasma cells are grown as dispersed populations of plasma cells overlayed by a monolayer of non-human mammary cells. In certain embodiments, the plasma cells are stimulated to produce immunoglobins during co-culture with non-human mammary cells.

In some embodiments, the plasma cells are derived from bone marrow, spleen, and/or a lymph node. a primary mammary tissue sample. In certain embodiments, the plasma cells are derived from mucosal epithelial cells other than mammary cells (e.g., from oronasal, gastrointestinal, or respiratory tissue). In some embodiments, the plasma cells are derived from a plasma cell line. In certain embodiments, the plasma cells are derived from a plasmacyte cell line. In some embodiments, the plasma cells are isolated and sorted from non-plasma cells via fluorescence-activated cell sorting, magnetic-activated cell sorting, and/or microfluidic cell sorting. In some embodiments, plasma cells, plasmablasts, or non-human mammal pre-plasmablasts are sorted and isolated by FACS analysis using markers known in the art (e.g., CD38, CD138 and/or CD19).

In some embodiments, the plasma cells are human plasma cells and the mammary cells are non-human mammary cells, to yield, for example, a cow milk product with human sIgA, a buffalo milk product with human sIgA, a bison milk product with human sIgA, a yak milk product with human sIgA, a camel milk product with human sIgA, a reindeer milk product with human sIgA, a goat milk product with human sIgA, or a sheep milk product with human sIgA.

In some embodiments, the plasma cells are cow plasma cells. In some embodiments, the plasma cells are goat plasma cells. In some embodiments, the plasma cells are sheep plasma cells. In some embodiments, the plasma cells are pig plasma cells. In some embodiments, the plasma cells are horse plasma cells. In some embodiments, the plasma cells are dog plasma cells. In some embodiments, the plasma cells are cat plasma cells.

### Scaffolds

In some embodiments, the cell construct further comprises a scaffold having a top surface/exterior surface and a bottom surface/interior surface. In some embodiments, the scaffold is a 2-dimensional surface or a 3-dimensional surface (e.g., a 3-dimensional micropatterned surface, and/or as a cylindrical structure that is assembled into bundles). A non-limiting example of a 2-dimensional surface scaffold is a Transwell® filter. In some embodiments, the scaffold is a 3-dimensional surface. Non-limiting examples of a 3-dimensional micropatterned surface include a microstructured bioreactor, a decellularized tissue (e.g., a decellularized mammary gland or decellularized plant tissue), micropatterned scaffolds fabricated through casting or three-dimensional printing with biological or biocompatible materials, textured surface. In some embodiments, the scaffold is produced by electrospinning cellulose nanofibers and/or a cylindrical structure that can be assembled into bundles (e.g., a hollow fiber bioreactor). In some embodiments, the scaffold is porous. In some embodiments, the scaffold is a 3D scaffold. In some embodiments, the 3-dimensional scaffold is any structure which has an enclosed hollow interior/central cavity. In some embodiments, the three-dimensional scaffold joins with one or more surfaces to form an enclosed interior chamber/basal compartment. For example, the scaffold can join with one or more walls of a bioreactor to form the interior chamber/basal compartment. In some embodiments, the scaffold is a hollow fiber bioreactor. In some embodiments, the 3D scaffold is a tube in which the central cavity is defined by the interior surface of the scaffold. In some embodiments, the 3D scaffold is a hollow sphere in which the central cavity is defined by the interior surface of the scaffold.

For in vitro culture methods for studies of intestinal absorption, 2-dimensional surface scaffold such as Transwells® have long been used as the standard as they provide both apical and basolateral spaces to simulate the gut-blood-barrier and enable both active and passive transport of drugs and nutrients. However, cells seeded onto flat supports exhibit markedly different phenotypes to cells in vivo, partly due to the poor representation of the 3-D extracellular microenvironments.

A 3-dimensional scaffold allows the cells (e.g., MECs and plasma cells) to grow or interact with their surroundings in all three dimensions. Unlike 2D environments, a 3D cell culture allows cells in vitro to grow in all directions, approximating the in vivo mammary environment. Further, the 3D scaffold allows for a larger surface area for culture of the cells and for metabolite and gas exchange, plus it enables necessary compartmentalization—enabling the milk product to be secreted into one compartment, while the cell culture media is contacted with the mammary cells and plasma cells in another compartment. To date, a confluent monolayer with polarized separation of basal and apical cell surfaces using mammary epithelial cell on a 3D surface has not been achieved (Sharfstein et al. 1992).

In some embodiments, the scaffold is porous. In some embodiments, the scaffold is permeable to the cell media, allowing the cell media to contact the cells of the cell monolayer. In some embodiments, the scaffold is transversed by at least one pore that allows the cell media to contact the basal surface of the cells of the cell monolayer.

In some embodiments, the top surface/exterior surface of the scaffold is coated with a matrix material. In some embodiments, the matrix is made up of one or more extracellular matrix proteins. Non-limiting examples of extracellular matrix proteins include collagen, laminin, entactin, tenascin, and/or fibronectin. In some embodiments, the scaffold comprises a natural polymer, a biocompatible synthetic polymer, a synthetic peptide, and/or a composite derived from any combination thereof. In some embodiments, a natural polymer useful with this invention includes, but is not limited to, collagen, chitosan, cellulose, agarose, alginate, gelatin, elastin, heparan sulfate, chondroitin sulfate, keratan sulfate, and/or hyaluronic acid. In some embodiments, a biocompatible synthetic polymer useful with this invention includes, but is not limited to, cellulose, polysulfone, polyvinylidene fluoride, polyethylene co-vinyl acetate, polyvinyl alcohol, sodium polyacrylate, an acrylate polymer, and/or polyethylene glycol. In some embodiments, the top of the scaffold is coated with laminin and collagen.

In some embodiments, the matrix material is porous. In some embodiments, the matrix material is permeable to the cell media, allowing the cell media to contact the cells of the cell monolayer. In some embodiments, the matrix material is transversed by at least one pore that allows the cell media to contact the basal surface of the cells of the cell monolayer.

In some embodiments, the pore size of the scaffold and/or matrix material is at least about 0.1 μm. In some embodiments, the pore size of the scaffold and/or matrix material is at least about 0.2 μm. In some embodiments, the pore size of the scaffold and/or matrix material is at least about 0.3 μm. In some embodiments, the pore size of the scaffold and/or matrix material is at least about 0.4 μm. In some embodiments, the pore size of the scaffold and/or matrix material is at least about 0.5 μm. In some embodiments, the pore size of the scaffold and/or matrix material is at least about 0.6 μm. In some embodiments, the pore size of the scaffold and/or matrix material is at least about 0.7 μm. In some embodiments, the pore size of the scaffold and/or matrix material is at least about 0.8 μm. In some embodiments, the pore size of the scaffold and/or matrix material is at least about 0.9 μm. In some embodiments, the pore size of the scaffold and/or matrix material is at least about 1.0 μm. In some embodiments, the pore size of the scaffold and/or matrix material is at least about 1.1 μm. In some embodiments, the pore size of the scaffold and/or matrix material is at least about 1.2 μm. In some embodiments, the pore size of the scaffold and/or matrix material is at least about 1.3 μm. In some embodiments, the pore size of the scaffold and/or matrix material is at least about 1.4 μm. In some embodiments, the pore size of the scaffold and/or matrix material is at least about 1.5 μm. In some embodiments, the pore size of the scaffold and/or matrix material is at least about 1.6 μm. In some embodiments, the pore size of the scaffold and/or matrix material is at least about 1.7 μm. In some embodiments, the pore size of the scaffold and/or matrix material is at least about 1.8 μm. In some embodiments, the pore size of the scaffold and/or matrix material is at least about 1.9 μm. In some embodiments, the pore size of the scaffold and/or matrix material is at least about 2.0 μm. In some embodiments, the pore size of the scaffold and/or matrix material is at least about 2.1 μm. In some embodiments, the pore size of the scaffold and/or matrix material is at least about 2.2 μm. In some embodiments, the pore size of the scaffold and/or matrix material is at least about 2.2 μm. In some embodiments, the pore size of the scaffold and/or matrix material is at least about 2.3 μm. In some embodiments, the pore size of the scaffold and/or matrix material is at least about 2.4 μm. In some embodiments, the pore size of the scaffold and/or matrix material is at least about 2.5 μm. In some embodiments, the pore size of the scaffold and/or matrix material is at least about 2.6 μm. In some embodiments, the pore size of the scaffold and/or matrix material is at least about 2.7 μm. In some embodiments, the pore size of the scaffold and/or matrix material is at least about 2.8 μm. In some embodiments, the pore size of the scaffold and/or matrix material is at least about 2.9 μm. In some embodiments, the pore size of the scaffold and/or matrix material is at least about 3.0 μm.

In some embodiments, the cell construct comprises: (a) a three dimensional scaffold having an exterior surface, an interior surface defining an interior cavity/basal chamber, and a plurality of pores extending from the interior surface to the exterior surface; (b) a matrix material disposed on the exterior surface of the three-dimensional scaffold; (c) a culture media disposed within the interior cavity/basal chamber and in fluidic contact with the internal surface; (d) a population of plasma cells (PCs) disposed on the matrix material and (e) a continuous monolayer of non-human mammary cells disposed on the population of plasma cells, the non-human mammary cells selected from the group consisting of: (i) non-human mammary epithelial cells, (ii) non-human mammary myoepithelial cells, and (iii) non-human mammary progenitor cells; wherein the continuous monolayer non-human mammary epithelial cells has an apical surface and a basal surface (e.g., the cells form a polarized and confluent cell monolayer).

### Bioreactor

Disclosed herein, in certain embodiments, are bioreactors, comprising: (a) an apical compartment comprising a milk product; and (b) at least one cell construct comprising: (a) a three dimensional scaffold having an exterior surface, an interior surface defining an interior cavity/basal chamber, and a plurality of pores extending from the interior surface to the exterior surface; (b) a matrix material disposed on the exterior surface of the three-dimensional scaffold; (c) a culture media disposed within the interior cavity/basal chamber and in fluidic contact with the internal surface; (d) a population of plasma cells (PCs) disposed on the matrix material and (e) a continuous monolayer of non-human mammary cells disposed on the population of plasma cells, the non-human mammary cells selected from the group consisting of: (i) non-human mammary epithelial cells, (ii) non-human mammary myoepithelial cells, and (iii) non-human mammary progenitor cells. In certain embodiments, the cell construct of the bioreactor comprises at least a 70% confluent monolayer of polarized non-human mammary cells disposed on the matrix material, wherein the non-human mammary cells are selected from the group consisting of: non-human mammary epithelial cells, non-human mammary myoepithelial cells, and non-human mammary progenitor cells; wherein the apical surface of the non-human mammary cells is in fluidic contact with the apical compartment.

In some embodiments, the bioreactor is an enclosed bioreactor. In some embodiments, the apical chamber is substantially isolated from the interior cavity/basal compartment.

A hollow fiber bioreactor is an exemplary bioreactor for use with the methods disclosed here. The hollow fiber bioreactor is a high-density, continuous perfusion culture system that closely approximates the environment in which cells grow in vivo. It consists of thousands of semi-permeable 3D scaffolds (i.e., hollow fibers) in a parallel array within a cartridge shell fitted with inlet and outlet ports. These fiber bundles are potted or sealed at each end so that any liquid entering the ends of the cartridge will necessarily flow through the interior of the fibers. Cells are generally seeded outside the fibers within the cartridge in the extra capillary space (ECS).

Three fundamental characteristics differentiate hollow fiber cell culture from other methods: (1) cells are bound to a porous matrix much as they are in vivo, not a plastic dish, microcarrier or other impermeable support, (2) the molecular weight cut off of the support matrix can be controlled, and (3) extremely high surface area to volume ratio (150 cm² or more per mL) which provides a large area for metabolite and gas exchange for efficient growth of host cells.

The bioreactor structure provides a fiber matrix that allows permeation of nutrients, gases and other basic media components, as well as cell waste products, but not cells, where the cells can be amplified. Hollow fiber bioreactor technology has been used to obtain high density cell amplification by utilizing hollow fibers to create a semi-permeable barrier between the cell growth chamber and the medium flow. Since the surface area provided by this design is large, using this fiber as a culture substrate allows the production of large numbers of cells. Cells growing in the 3-dimensional environment within the bioreactor are bathed in fresh medium as it perfuses through the hollow fibers.

To replicate the topography of the intestine, Costello et al. developed a 3-D printed bioreactor that can both contain porous villus scaffolds via micromolding (Costello et al. 2017 Scientific Reports 7(12515): 1-10). This geometrically complex molded scaffold provided separation of the apical and basolateral spaces in a manner in which fluid flow exposes intestinal epithelial cells to physiologically relevant shear stresses (Costello et al. 2017). Similarly, a long-term culture in vitro culture in a simulated gut-like environment was created by Morada et al. using a hollow fiber bioreactor which allowed for two controlled separate environments (biphasic) to provide host cells with oxygen and nutrients from the basal layer, while allowing a low oxygen nutrient rich environment to be developed on the apical surface (Morada et al. 2016 International Journal for Parasitology 26: 21-29).

In configuring the hollow fiber bioreactor, there are design considerations and parameters that can be varied depending upon the goals associated with expansion of the cells. One such design consideration is the size of the pores in the fiber wall. This is generally designed to allow the passage of nutrients to the cells, carry away waste, provide desired products to the cells (such as growth factors), to remove desired products from the cells, and exclude certain factors that may be present from reaching the cells. Accordingly, the pore size of the fiber walls can be varied to modify which components will pass through the walls. For example, pore size can allow the passage of large proteinaceous molecules, including growth factors, including, but not limited to, epidermal growth factor and platelet-derived growth factor. The person of ordinary skill in the art would understand how to vary the pore size depending upon the components that it is desirable to pass through the fiber walls to reach the cells or to carry material from the cells.

In some embodiments, the pore size is about 0.2 μm. In some embodiments, the pore size is about 0.1. In some embodiments, the pore size is about 0.2 μm. In some embodiments, the pore size is about 0.3 μm. In some embodiments, the pore size is about 0.4 μm. In some embodiments, the pore size is about 0.5 μm. In some embodiments, the pore size is about 0.6 μm. In some embodiments, the pore size is about 0.7 μm. In some embodiments, the pore size is about 0.8 μm. In some embodiments, the pore size is about 0.9 μm. In some embodiments, the pore size is about 1.0 μm. In some embodiments, the pore size is about 1.1 μm. In some embodiments, the pore size is about 1.2 μm. In some embodiments, the pore size is about 1.3 μm. In some embodiments, the pore size is about 1.4 μm. In some embodiments, the pore size is about 1.5 μm. In some embodiments, the pore size is about 1.6 μm. In some embodiments, the pore size is about 1.7 μm. In some embodiments, the pore size is about 1.8 μm. In some embodiments, the pore size is about 1.9 μm. In some embodiments, the pore size is about 2.0 μm. In some embodiments, the pore size is about 2.1 μm. In some embodiments, the pore size is about 2.2 μm. In some embodiments, the pore size is about 2.2 μm. In some embodiments, the pore size is about 2.3 μm. In some embodiments, the pore size is about 2.4 μm. In some embodiments, the pore size is about 2.5 μm. In some embodiments, the pore size is about 2.6 μm. In some embodiments, the pore size is about 2.7 μm. In some embodiments, the pore size is about 2.8 μm. In some embodiments, the pore size is about 2.9 μm. In some embodiments, the pore size is about 3.0 μm.

### Methods of Making Cell Constructs

Disclosed herein, in certain embodiments, are methods of making a cell construct for producing a non-human milk product comprising immunoglobulins. In some embodiments, the method comprises (a) depositing (i) isolated non-human mammary epithelial cells (MECs), non-human mammary myoepithelial cells and/or non-human mammary progenitor cells, and (ii) isolated plasma cells on the upper surface of a scaffold having an upper surface and lower surface to produce a mixed population of plasma cells and non-human mammary cells (i.e., non-human mammary epithelial cells, non-human mammary myoepithelial cells and/or non-human mammary progenitor cells); (b) cultivating the mixed population of non-human mammary cells and plasma cells of (a) on the scaffold, to produce a monolayer of polarized non-human mammary cells located adjacent to and above the plasma cells, wherein the plasma cells are located adjacent to and above the upper surface of the scaffold, wherein the upper surface is located adjacent to and above the lower surface of the scaffold, and wherein the polarized non-human mammary cells comprise an apical surface and a basal surface, thereby producing a cell construct for producing the non-human mammal milk product. In some embodiments, the non-human mammary cells are primary non-human mammary cells. In some embodiments, the non-human mammary cells are derived from a cell culture. In some embodiments, the non-human mammary epithelial cells, non-human mammary myoepithelial cells and/or non-human mammary progenitor cells are isolated from bone marrow, spleen tissue, lymph node tissue, non-human mammary explants from non-human mammary tissue, or raw milk. In some embodiments, the non-human mammary cells comprise non-human mammary epithelial cells. In some embodiments, the non-human mammary cells, comprise non-human mammary myoepithelial cells. In some embodiments, the non-human mammary cells, comprise non-human mammary progenitor cells. In some embodiments, the plasma cells are isolated from any suitable tissue or a cell culture. In some embodiments, the non-human mammary cells and plasma cells are deposited concurrently. In some embodiments, the plasma cells are deposited onto the surface of the scaffold prior to the deposition of the non-human mammary cells.

In some embodiments, the method comprises (a) depositing (i) isolated immortalized non-human mammary epithelial cells, non-human mammary myoepithelial cells and/or non-human mammary progenitor cells, and (ii) isolated plasma cells on the upper surface of a scaffold having an upper surface and lower surface to produce a mixed population of plasma cells and immortalized non-human mammary cells (i.e., immortalized non-human mammary epithelial cells, immortalized non-human mammary myoepithelial cells and/or immortalized non-human mammary progenitor cells); (b) cultivating the mixed population of immortalized non-human mammary cells and plasma cells of (a) on the scaffold, to produce a monolayer of polarized immortalized non-human mammary cells located adjacent to and above the plasma cells, wherein the plasma cells are located adjacent to and above the upper surface of the scaffold, wherein the upper surface is located adjacent to and above the lower surface of the scaffold, and wherein the polarized immortalized non-human mammary cells comprise an apical surface and a basal surface, thereby producing a cell construct for producing the milk product. In some embodiments, the immortalized non-human mammary cells comprise immortalized non-human mammary epithelial cells. In some embodiments, the immortalized non-human mammary cells, comprise immortalized non-human mammary myoepithelial cells. In some embodiments, the immortalized non-human mammary cells, comprise immortalized non-human mammary progenitor cells. In some embodiments, the plasma cells are isolated from any suitable human tissue or a cell culture. In some embodiments, the immortalized non-human mammary cells and plasma cells are deposited concurrently. In some embodiments, the plasma cells are deposited onto the surface of the scaffold prior to the deposition of the immortalized non-human mammary cells. In certain embodiments, plasma cells are added to the culture of immortalized non-human mammary epithelial cells to produce a co-culture of non-human mammary cells and plasma cells. In certain embodiments, the plasma cells are cultivated with the immortalized non-human mammary epithelial cells on the scaffold, thereby producing a cell construct for producing a milk product with secretory products of the immune cells and non-human mammary cells (e.g., sIgA). In certain embodiments, the isolated non-human mammary cells are immortalized prior to co-culture of the cells.

In certain embodiments, the non-human mammal plasma cells are stimulated to produce immunoglobins during co-culture. In certain embodiments, the non-human mammal plasma cells produce one or more immunoglobins of a class selected from IgG, IgM and IgA. In certain embodiments the non-human mammal plasma cells produce secretory IgA. Classes of immunoglobins produced by the non-human mammal plasma cells include one or more IgA, IgM, and IgG. In certain embodiments, non-human mammal plasma cells are co-cultured with MECs in a bioreactor according to methods described herein. In certain embodiments, the bioreactor is a hollow fiber bioreactor described herein.

In certain embodiments, non-human mammary cells are modified and/or stimulated with prolactin according to the methods described herein to stimulate and optimize milk production. In certain embodiments, the non-human mammary cells are modified to express a constitutively active prolactin receptor protein.

In certain embodiments, non-human mammary cells are identified and isolated from non-human mammary tissue samples. In some embodiments, the non-human mammary cells are isolated and sorted via fluorescence-activated cell sorting, magnetic-activated cell sorting, and/or microfluidic cell sorting. In certain embodiments, the non-human mammary epithelial cell populations are sorted by FACS analysis using markers known in the art for identifying the cell populations. In certain embodiments, myoepithelial non-human mammary cells and luminal epithelial non-human mammary cells are isolated by FACS analysis. In certain embodiments, progenitor myoepithelial non-human mammary cells and/or progenitor luminal epithelial non-human mammary cells are isolated by FACS analysis. Any suitable method known in the art for sorting non-human mammary epithelial cells (e.g., luminal epithelial cells), myoepithelial cells, progenitor cells, and immune cells can be used.

In some embodiments, plasma cells are identified and isolated from primary mucosal tissue (e.g., oronasal, gastrointestinal, respiratory or non-human mammary). In some embodiments, plasma cells are identified and isolated from primary non-human mammary tissue samples. In some embodiments, the plasma cells are isolated and sorted via fluorescence-activated cell sorting, magnetic-activated cell sorting, and/or microfluidic cell sorting. In certain embodiments, plasma cells are sorted and isolated by FACS analysis. In certain embodiments plasma cells, plasmablasts, or pre-plasmablasts are sorted and isolated by FACS analysis using markers known in the art (e.g., CD20, CD38, CD138 and/or CD19).

In some embodiments, the cell construct comprises a scaffold comprising an upper surface and a lower surface and a continuous monolayer of polarized non-human mammary epithelial cells, a continuous monolayer of a polarized, mixed population of non-human mammary epithelial cells, non-human mammary myoepithelial cells and non-human mammary progenitor cells, and/or a continuous monolayer of polarized immortalized non-human mammary epithelial cells, wherein the continuous monolayer is located on the upper surface of scaffold.

In some embodiments, the lower surface of the scaffold is adjacent to the basal compartment. In some embodiments, the apical surface of the continuous monolayer is adjacent to the apical compartment. In some embodiments, the continuous monolayer secretes milk and sIgA or IgA through its apical surface into the apical compartment, thereby producing a milk product comprising IgA and/or sIgA in culture.

In some embodiments, the monolayer of non-human mammary cells forms a barrier that divides the apical compartment and the basal compartment, wherein the basal surface of the non-human mammary cells is attached to the scaffold and the apical surface is oriented toward the apical compartment.

In some embodiments, the basal compartment is adjacent to the lower surface of the scaffold. In some embodiments, the basal compartment comprises a culture medium in fluidic contact with the basal surface of the monolayer of non-human mammary epithelial cells (e.g., the polarized monolayer of non-human mammary epithelial cells, the polarized the monolayer of the mixed population of non-human mammary cells, or the polarized monolayer of immortalized non-human mammary epithelial cells).

In some embodiments, the culture medium comprises a carbon source, a chemical buffering system, one or more essential amino acids, one or more vitamins and/or cofactors, and one or more inorganic salts.

In some embodiments, the bioreactor comprises an apical compartment that is adjacent to the apical surface of the monolayer. In some embodiments, the apical compartment is adjacent to the upper surface of the scaffold.

In some embodiments, the total cell density of non-human mammary cells in the bioreactor is at least 10¹¹ non-human mammary cells. In some embodiments, the total cell density of non-human mammary cells in the bioreactor is at least 10¹² non-human mammary cells. In some embodiments, the total cell density of non-human mammary cells in the bioreactor is at least 10¹³ non-human mammary cells.

In some embodiments, the total cell density of non-human mammary cells in the bioreactor is about 20 to 55 cells per 100 μm². In some embodiments, the total cell density of non-human mammary cells in the bioreactor is about 20 cells per 100 μm². In some embodiments the total cell density of non-human mammary cells in the bioreactor is 25 cells per 100 μm². In some embodiments, the total cell density of non-human mammary cells in the bioreactor is about 30 cells per 100 μm². In some embodiments, the total cell density of non-human mammary cells in the bioreactor is about 35 cells per 100 μm². In some embodiments, the total cell density of non-human mammary cells in the bioreactor is about 40 cells per 100 μm². In some embodiments, the total cell density of non-human mammary cells in the bioreactor is about 45 cells per 100 μm². In some embodiments, the total cell density of non-human mammary cells in the bioreactor is about 50 cells per 100 μm². In some embodiments, the total cell density of non-human mammary cells in the bioreactor is about 55 cells per 100 μm².

In some embodiments, the total cell density of plasma cells in the bioreactor is about 200 to 500 plasma cells per mm². In some embodiments, the total cell density of plasma cells in the bioreactor is about 200 plasma cells per mm². In some embodiments, the total cell density of plasma cells in the bioreactor is about 300 plasma cells per mm². In some embodiments, the total cell density of plasma cells in the bioreactor is about 400 plasma cells per mm². In some embodiments, the total cell density of plasma cells in the bioreactor is about 500 plasma cells per mm².

In some embodiments, the total surface area of non-human mammary cells within the bioreactor is at least about 1.5 m². In some embodiments, the total surface area of non-human mammary cells within the bioreactor is at least about 2 m². In some embodiments, the total surface area of non-human mammary cells within the bioreactor is at least about 2.5 m². In some embodiments, the total surface area of non-human mammary cells within the bioreactor is at least about 3 m². In some embodiments, the total surface area of non-human mammary cells within the bioreactor is at least about 4 m². In some embodiments, the total surface area of non-human mammary cells within the bioreactor is at least about 5 m². In some embodiments, the total surface area of non-human mammary cells within the bioreactor is at least about 10 m². In some embodiments, the total surface area of non-human mammary cells within the bioreactor is at least about 15 m². In some embodiments, the total surface area of non-human mammary cells within the bioreactor is at least about 20 m². In some embodiments, the total surface area of non-human mammary cells within the bioreactor is at least about 25 m². In some embodiments, the total surface area of non-human mammary cells within the bioreactor is at least about 50 m². In some embodiments, the total surface area of non-human mammary cells within the bioreactor is at least about 100 m². In some embodiments, the total surface area of non-human mammary cells within the bioreactor is at least about 250 m². In some embodiments, the total surface area of non-human mammary cells within the bioreactor is at least about 500 m².

In some embodiments, the bioreactor maintains a temperature of about 27° C. to about 39° C. (e.g., a temperature of about 27° C., 28° C., 29° C., 30° C., 31° C., 32° C., 33° C., 34° C., 35° C., 35° C., 35.5° C., 36° C., 36.5° C., 37° C., 37.5° C., 38° C., 38.5° C. or about 39° C., or any value or range therein, e.g., about 27° C. to about 38° C., about 36° C. to about 39° C., about 36.5° C. to about 39° C., about 36.5° C. to about 37.5° C., or about 36.5° C. to about 38° C.). In some embodiments, the bioreactor maintains a temperature of about 37° C.

In some embodiments, the bioreactor has an atmospheric concentration of CO₂ of about 4% to about 6%, e.g., an atmospheric concentration of CO2 of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the bioreactor has an atmospheric concentration of CO₂ of about 5%.

In some embodiments, the bioreactor has an atmospheric concentration of CO₂ of about 4% to about 6%, e.g., an atmospheric concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the bioreactor has an atmospheric concentration of CO₂ of about 5%.

In some embodiments, the method comprises monitoring the concentration of dissolved O₂ and CO₂. In some embodiments, the concentration of dissolved O₂ is maintained between about 10% to about 25% or any value or range therein (e.g., about 10, 11, 12, 13, 14, 15, 16, 17, 18, 19, 20, 21, 22, 23, 24, or 25%). For example, in some embodiments, the concentration of dissolved O₂ is maintained between about 12% to about 25%, about 15% to about 22%, about 10% to about 20%, about 15%, about 20%, or about 22%. In some embodiments, the concentration of CO₂ is maintained between about 4% to about 6%, e.g., a concentration of CO₂ of about 4%, 4.25%, 4.5%, 4.75%, 5%, 5.25%, 5.5%, 5.75%, or 6% or any value or range therein, e.g., about 4% to about 5.5%, about 4.5% to about 6%, about 4.5% to about 5.5%, or about 5% to about 6%). In some embodiments, the concentration of CO₂ is maintained at about 5%.

In some embodiments, the culture medium is exchanged about every day to about every 10 days (e.g., every 1 day, every 2 days, every 3 days, every 4 days, every 5 days, every 6 days, every 7 days, every 8 days, every 9 days, every 10 days, or any value or range therein, e.g., about every day to every 3 days, about every 3 days to every 10 days, about every 2 days to every 5 days). In some embodiments, the culture medium is exchanged about every day to about every few hours to about every 10 days, e.g., about every 1, 2, 3, 4, 5, 6, 7, 8, 9, 10, 11, 12, 13, 14, 15, 16, 17, 18, 19, 20, 21, 22, 23, or 24 hours to about every 1, 2, 3, 4, 5, 6, 7, 8, 9, or 10 days or any value or range therein. For example, in some embodiments, the culture medium is exchanged about every 12 hours to about every 10 days, about every 10 hours to about every 5 days, or about every 5 hours to about every 3 days.

In some embodiments, the method comprises monitoring the glucose concentration and/or rate of glucose consumption in the culture medium and/or in the lactogenic culture medium. In some embodiments, the prolactin is added when the rate of glucose consumption in the culture medium is steady state.

In some embodiments, the method further comprises applying transepithelial electrical resistance (TEER) to measure the maintenance of the monolayer of epithelial cells. TEER measures a voltage difference between the fluids (e.g., media) in two compartments (e.g., between the apical and basal compartments), wherein if the barrier between the compartments loses integrity, the fluids in the two compartments may mix. When there is fluid mixing, the voltage difference will be reduced or eliminated; a voltage difference indicates that the barrier is intact. In some embodiments, upon detection of a loss of voltage by TEER, a scaffold (e.g., a Transwell® filter, a microstructured bioreactor, a decellularized tissue, a hollow fiber bioreactor, etc.) is reinoculated with additional cells and allowed time to reestablish a barrier (e.g., a monolayer) before resuming production of the cultured milk product (e.g., milk production).

In some embodiments, the method further comprises collecting the milk product from the apical compartment to produce collected milk product. In some embodiments, the collecting is via a port, via gravity, and/or via a vacuum. In some embodiments, a vacuum is attached to a port.

In some embodiments, the method further comprises freezing the collected milk product to produce frozen cultured milk product and/or lyophilizing the collected milk product to produce lyophilized cultured milk product.

In some embodiments, the method further comprises packaging the collected milk product, the frozen milk product and/or the lyophilized milk product into a container.

In some embodiments, the method further comprises extracting one or more components from the collected milk product. Non-limiting examples of components from the collected milk product include milk protein, lipid, carbohydrate, vitamin, and/or mineral contents. In some embodiments, the components from the collected milk product are lyophilized and/or concentrated to produce a lyophilized or a concentrated milk product component product. In some embodiments, the components from the collected milk product are concentrated by, e.g., membrane filtration and/or reverse osmosis. In some embodiments, the lyophilized or concentrated milk product component product is packaged in a container, optionally wherein the container is sterile and/or a food grade container. In some embodiments, the container is vacuum-sealed. In some embodiments, the container is a canister, ajar, a bottle, a bag, a box, or a pouch. In some embodiments, the milk product is a standardized, sterile milk product. In some embodiments, the milk product is for nutritional use.

In some embodiments, the milk product is produced by any method disclosed herein.

### Non-Human Milk Products

Disclosed herein, in certain embodiments, are non-human milk products wherein the milk product does not comprise or is substantially free of persistent organic pollutants (POPs), heavy metals, non-milk allergens, cells, hormones, or virus; provided that the cultured milk product may comprise a mammary epithelial cell (hMEC) or a plasma cell (PC). In some embodiments, the non-human milk product is produced by any method disclosed herein.

In some embodiments, the milk product is a cow milk product. In some embodiments, the milk product is a cow milk product and does not comprise beta-lactoglobulin. In some embodiments, the milk product is a bison milk product. In some embodiments, the milk product is a buffalo milk product. In some embodiments, the milk product is a yak milk product. In some embodiments, the milk product is a goat milk product. In some embodiments, the milk product is a sheep milk product. In some embodiments, the milk product is a camel milk product. In some embodiments, the milk product is a reindeer milk product. In some embodiments, the milk product is a pig milk product. In some embodiments, the milk product is a dog milk product. In some embodiments, the milk product is a cat milk product. In some embodiments, the milk product is a horse milk product.

### Immunoglobulins

In some embodiments, the non-human milk product further comprises one or more immunoglobulins or sIgA. In some embodiments, the non-human milk product comprises one or more of IgA, IgG, and IgM. In some embodiments, the non-human milk product comprises IgA2 (secretory) and IgA1 (non-secretory).

In some embodiments, the non-human milk product comprises one or more human immunoglobulins or human sIgA. In some embodiments, the non-human milk product comprises one or more of human IgA, human IgG, and human IgM. In some embodiments, the non-human milk product comprises human IgA2 (secretory) and human IgA1 (non-secretory).

In some embodiments, the non-human milk product comprises about 0.2-1.0 g/L secretory IgA. In some embodiments, the non-human milk product comprises about 0.2, 0.25, 0.3, 0.35, 0.4, 0.45, 0.5, 0.55, 0.6, 0.65, 0.7, 0.75, 0.8, 0.85, 0.9, 0.95, or 1.0 g/L secretory IgA. In some embodiments, the non-human milk product comprises about 0.15-1.6 g/L total IgA. In some embodiments, the non-human milk product comprises about 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, or 1.6 g/L total IgA.

In some embodiments that comprise IgG, the non-human milk product comprises about 0.03-0.3 g/L IgG. In some embodiments, that comprise IgG, the non-human milk product comprises about 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3 g/L. In some embodiments that comprise IgM, the non-human milk product comprises about 0.01-0.1 g/L IgM. In some embodiments, the non-human milk product comprises about 0.01, 0.02, 0.03, 0.04, 0.05, 0.06, 0.07, 0.08, 0.09, 0.1, 0.1 g/L IgM. In some embodiments, the non-human milk product comprises about 0.2-2.0 percent by weight total immunoglobulins. In certain embodiments, the non-human milk product comprises 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 percent by weight total immunoglobulins.

Isolated sIgA Immunotherapeutic Compositions

Disclosed herein, in certain embodiments, are immunotherapeutic compositions, comprising: (a) isolated non-human mammal IgA and/or non-human mammal sIgA, and (b) a pharmaceutically acceptable excipient. In some embodiments, the IgA or sIgA is derived from the secreted products of non-human mammary epithelial cells (MECs) co-cultured with non-human plasma cells. In some embodiments, the isolated non-human IgA and non-human sIgA are isolated from the product resulting from co-culturing non-human mammary epithelial cells (MECs) and non-human plasma cells (e.g., a milk product).

In some embodiments, the non-human IgA or non-human sIgA is bovine IgA or bovine sIgA. In some embodiments, the non-human IgA or non-human sIgA is bison IgA or bison sIgA. In some embodiments, the non-human IgA or non-human sIgA is buffalo IgA or buffalo sIgA. In some embodiments, the non-human IgA or non-human sIgA is yak IgA or yak sIgA. In some embodiments, the non-human IgA or non-human sIgA is goat IgA or goat sIgA. In some embodiments, the non-human IgA or non-human sIgA is sheep IgA or sheep sIgA. In some embodiments, the non-human IgA or non-human sIgA is camel IgA or camel sIgA. In some embodiments, the non-human IgA or non-human sIgA is reindeer IgA or reindeer sIgA. In some embodiments, the non-human IgA or non-human sIgA is pig IgA or pig sIgA. In some embodiments, the non-human IgA or non-human sIgA is dog IgA or dog sIgA. In some embodiments, the non-human IgA or non-human sIgA is cat IgA or cat sIgA. In some embodiments, the non-human IgA or non-human sIgA is horse IgA or horse sIgA.

In some embodiments, the pharmaceutically acceptable excipient is a stabilizer, a surfactant, a buffer or tonicity agent. In some embodiments, the pharmaceutically acceptable excipient is sucrose, trehalose, mannitol, sorbitol, histidine, arginine, glycine, polysorbate 20, polysorbate 80, poloxamer 188, edetic acid/or edetate salts (e.g., EDTA), glutathione, metacresol, phenol, benzyl alcohol, benzalkonium chloride, methionine or cysteine.

### Processing

In some embodiments, the milk product is sterilized. In some embodiments, the milk product is pasteurized. In some embodiments, the milk product is frozen. In some embodiments, the milk product is lyophilized. In some embodiments, the milk product is in a container.

### Excluded Components

By virtue of the fact that the milk products produced by the methods disclosed herein are manufactured outside of the body of the non-human mammal, the milk products are free of or substantially free of certain components found in naturally-occurring milk. In some cases, the components are harmful (e.g., environmental contaminants and allergens) and in other cases, the naturally occurring components are simply present due to the naturally occurring milk being naturally produced. Further, the design of the live cell construct and the bioreactor enables the production of milk products disclosed herein that are free of or substantially free of cell culture media and components thereof.

### Cell Culture Media

Certain components of cell culture media may pass into the milk product. For example, serum albumin, linoleic acid and alpha-linolenic acid may be actively secreted by the hMECs into the apical compartment with components of the milk product produced by the hMECs. In some embodiments, the total dry mass of the milk product disclosed herein comprises about 1% w/w to about 20% of cell media components greater than 150 kDa. In some embodiments, the total dry mass of the milk product disclosed herein comprises about 5% w/w to about 10% of cell media components greater than 150 kDa. In some embodiments, the total dry mass of the milk product disclosed herein comprises about 5% w/w of cell media components greater than 150 kDa. In some embodiments, the total dry mass of the milk product disclosed herein comprises about 20% w/w to about 40% of cell media components less than 150 kDa. In some embodiments, the total dry mass of the milk product disclosed herein comprises about 25% w/w to about 35% of cell media components less than 150 kDa. In some embodiments, the total dry mass of the milk product disclosed herein comprises about 25% w/w to about 30% of cell media components less than 150 kDa. In some embodiments, the total dry mass of the milk product disclosed herein comprises about 25% w/w of cell media components less than 150 kDa.

In some embodiments, the total dry mass of the milk product disclosed herein comprises less than 80% w/w of cell media components greater than 150 kDa. In some embodiments, the total dry mass of the milk product disclosed herein comprises less than 85% w/w of cell media components greater than 150 kDa. In some embodiments, the total dry mass of the milk product disclosed herein comprises less than 90% w/w of cell media components greater than 150 kDa. In some embodiments, the total dry mass of the milk product disclosed herein comprises less than 95% w/w of cell media components greater than 150 kDa. In some embodiments, the total dry mass of the milk product disclosed herein comprises less than 97% w/w of cell media components greater than 150 kDa. In some embodiments, the total dry mass of the milk product disclosed herein comprises less than 98% w/w of cell media components greater than 150 kDa. In some embodiments, the total dry mass of the milk product disclosed herein comprises less than 99% w/w of cell media components greater than 150 kDa.

In some embodiments, the total dry mass of the milk product disclosed herein comprises less than 60% w/w of cell media components less than 150 kDa. In some embodiments, the total dry mass of the milk product disclosed herein comprises less than 65% w/w of cell media components less than 150 kDa. In some embodiments, the total dry mass of the milk product disclosed herein comprises less than 70% w/w of cell media components less than 150 kDa. In some embodiments, the total dry mass of the milk product disclosed herein comprises less than 75% w/w of cell media components less than 150 kDa. In some embodiments, the total dry mass of the milk product disclosed herein comprises less than 77% w/w of cell media components less than 150 kDa. In some embodiments, the total dry mass of the milk product disclosed herein comprises less than 78% w/w of cell media components less than 150 kDa. In some embodiments, the total dry mass of the milk product disclosed herein comprises less than 79% w/w of cell media components less than 150 kDa. In some embodiments, the total dry mass of the milk product disclosed herein comprises less than 80% w/w of cell media components less than 150 kDa.

### Environmental Contaminants and Drugs

Milk contains low but measurable concentrations of environmental contaminants, health-harming chemicals from industry and manufacturing products that are widely spread in the environment. Environmental contaminants are partly secreted in milk. Persistent organic pollutants (POPs) are a family of lipophilic stable chemicals that bioaccumulate in adipose tissue and create a lasting toxic body burden.

In some embodiments, the milk product does not comprise or is substantially free of one or more environmental contaminants. In some embodiments, the milk product does not comprise or is substantially free of persistent organic pollutants (POPs). In some embodiments, the milk product does not comprise or is substantially free of polychlorinated dibenzo-p-dioxins (PCDDs), polychlorinated dibenzofurans (PCDFs), polychlorinated biphenyls (PCBs) and pesticides such as DDT.

Heavy metals such as mercury, lead, arsenic, cadmium, nickel, chromium, cobalt, zinc, and other potentially toxic metals that are dispersed throughout the environment also have bioaccumulative features known to accumulate in milk. Metal in milk originates from exogenous sources, i.e., uptake via contaminated air, food (e.g., grass, hay), and drinking water, and endogenous release along with essential trace elements. For example, lead and mercury are equally dispersed in the environments. The exposures to toxic metals have significant public health implication. Even at small concentrations and acute exposures, these metals remain toxic to humans and non-human mammals.

In some embodiments, the milk product does not comprise or is substantially free of one or more heavy metals, such as arsenic, lead, cadmium, nickel, mercury, chromium, cobalt, and zinc. In some embodiments, the milk product does not comprise or is substantially free of arsenic. In some embodiments, the milk product does not comprise or is substantially free of lead. In some embodiments, the milk product does not comprise or is substantially free of cadmium. In some embodiments, the milk product does not comprise or is substantially free of nickel. In some embodiments, the milk product does not comprise or is substantially free of mercury. In some embodiments, the milk product does not comprise or is substantially free of chromium. In some embodiments, the milk product does not comprise or is substantially free of cobalt. In some embodiments, the milk product does not comprise or is substantially free of zinc. In some embodiments, the milk product does not comprise or is substantially free of arsenic, lead, cadmium, nickel, mercury, chromium, cobalt, and zinc.

### Cells

Naturally occurring milk contains a substantial population of living cells representing a diversity of cell types, including stem cells, immune cells, and bacterial cells. Bacteria found in the milk of various animals include Staphylococcus, Acinetobacter, Streptococcus, Pseudomonas, Lactococcus, Enterococcus, Lactobacillus, Mycobacterium bovis, Brucellosis, Listeria, and Campylobacter.

In some embodiments, the milk product does not comprise or is substantially free of a cell. In some embodiments, the milk product comprises a human mammary epithelial cell and/or a plasma cell. In some embodiments, the milk product does not comprise or is substantially free of a cell other than one or more human mammary epithelial cells and/or one or more plasma cells. In some embodiments, the milk product does not comprise or is substantially free of cells selected from the group consisting of: stem cells, myoepithelial cells, myeloid precursor cells, neutrophils, granulocytes, T cells, Staphylococcus, Acinetobacter, Streptococcus, Pseudomonas, Lactococcus, Enterococcus, Lactobacillus, Mycobacterium bovis, Brucellosis, Listeria, and Campylobacter. In some embodiments, the milk product comprises a mammary epithelial cell and/or a plasma cell. In some embodiments, the milk product does not comprise or is substantially free of a cell other than one or more non-human mammary epithelial cells (MECs) and/or one or more plasma cells.

### Viruses

Naturally occurring milk may transmit certain infectious agents, such as bovine leukemia virus, tick-borne encephalitis virus, bovine herpesvirus (BHV) 1, BHV2, BHV4, bovine viral diarrhea virus (BVDV). In some embodiments, the milk product does not comprise or is substantially free of a virus.

### Hormones

Naturally occurring milk may include hormones from maternal circulation, such as leptin, ghrelin, and adiponectin, that are synthesized outside the mammary gland and transported into milk across the mammary epithelium. In some embodiments, the milk product does not comprise a hormone. In some embodiments, the milk product does not comprise a hormone selected from the group consisting of: leptin, ghrelin, adiponectin, thyroxine (T4), triiodothyronine (T3) thyroid-stimulating hormone (TSH), epidermal growth factor, beta-endorphin, relaxin, cortisol, and erythropoietin.

### Exemplary Cow Milk Products

Cow milk and cow milk products are nutritious food items containing numerous essential nutrients such as, oleic acid, conjugated linoleic acid, omega-3 fatty acids, vitamins, minerals and bioactive compounds such as antioxidants. However, due to the extreme processes that cow milk goes through and the exposure of cows to antibiotics, hormones, genetic selection, change in diet, and genetically-modified substances, there are concerns associated with drinking milk from cows. Cows release contaminants and toxins through their milk, as milk is a natural exit-portal for substances that the body cannot use. Examples of potential contaminants of cow milk include hormones (e.g., pituitary, steroid, hypothalamic, and thyroid hormones), gastrointestinal peptides (e.g., nerve and epidermal growth factors, and the growth inhibitors MDGI and MAF), rBGH or recombinant cow growth hormone (a genetically engineered hormone injected into cows to increase milk production which has been linked to breast, colon and prostate cancer), pus from infected cow udders, and/or antibiotics or pharmaceuticals which have been administered to cows.

Cow milk also harbors a complex microbial community, including microorganisms that are of concern from a food quality or safety perspective. The cow milk microbiota is the focus of constant attention and testing. Such testing occurs daily on both raw and pasteurized cow milk. The microbial composition of milk is influenced by several different parameters such as, in the case of raw cow milk, the microorganisms present in the teat canal, on the surface of teat skin, in the surrounding air, in feed, as well as other environmental factors including cow housing conditions, the quality of the water supply, and equipment hygiene. The microbiota of pasteurized cow milk is thought to be determined by the percentage of thermoduric bacteria that survive pasteurization temperatures and by the bacteria associated with post-pasteurization contamination. It has been suggested that the potential for microbes, usually considered to be eliminated by pasteurization, to survive commercial pasteurization and the apparent presence of these populations in commercial milk, and there is a potential effect on milk quality, shelf-life, and milk-based products.

The present disclosure relates to milk product compositions that comprise protein, lipid, and oligosaccharide components and component concentrations that mimic cow milk, which compositions are produced, at least in part, by in vitro and/or ex vivo cultured cow mammary cells.

Contemplated milk product compositions of the present disclosure can be defined by total levels of protein, lipid, and carbohydrate (Tables 1A-1C) and/or by a signature of specific macronutrient components (Tables 2A-2C) present in concentrations and proportions consistent with cow milk.

TABLE 1A
Macromolecular Composition of Functional Nutrition Products
Collected from Cow Mammary Epithelial Cells.

| Macromolecular Fraction | Concentration, g/L |
|---|---|
| Protein | 14-60 |
| Lipid | 18-83 |
| Milk oligosaccharides | 0.23 |
| Lactose | 20-90 |
| Total macromolecular content | 50-235 |
| Energy (kcal/L) | 225-1350 |

Long-chain fatty acids linoleic acid and alpha-linoleic acid are not synthesized by mammalian cells and are supplemented in cell culture media.

TABLE 1B
Macromolecular Composition of Functional Nutrition Products
Collected from Cow Mammary Epithelial Cells.

| Macromolecular Fraction | Concentration, g/L |
|---|---|
| Protein | 21-50 |
| Lipid | 26-41 |
| Milk oligosaccharides | 0.008-0.19 |
| Lactose | 30-75 |
| Total macromolecular content | 75-170 |
| Energy (kcal/L) | 338-1125 |

Long-chain fatty acids linoleic acid and alpha-linoleic acid are not synthesized by mammalian cells and are supplemented in cell culture media.

TABLE 1C
Macromolecular Composition of Functional Nutrition Products
Collected from Cow Mammary Epithelial Cells.

| Macromolecular Fraction | Concentration, g/L |
|---|---|
| Protein | 28-40 |
| Lipid | 35-55 |
| Milk oligosaccharides | 0.01-0.15 |
| Lactose | 40-60 |
| Total macromolecular content | 100-160 |
| Energy (kcal/L) | 450-900 |

Long-chain fatty acids linoleic acid and alpha-linoleic acid are not synthesized by mammalian cells and are supplemented in cell culture media.

In some embodiments, the concentrations of components indicated in Tables 1A-1C can vary, each individually, for example, by having a concentration that is greater than that indicated by 0.1 fold, or 0.2 fold, or 0.3 fold, or 0.4 fold, or 0.5 fold, or 0.6 fold, or 0.7 fold, or 0.8 fold, or 0.9 fold, or 1, 2, 3, 4, 5, 6, 7, 8, 9, or 10 fold.

In some embodiments, the concentrations of components indicated in Tables 1A-1C can vary, each individually, for example, by having a concentration that is less than that indicated by 0.1 fold, or 0.2 fold, or 0.3 fold, or 0.4 fold, or 0.5 fold, or 0.6 fold, or 0.7 fold, or 0.8 fold, or 0.9 fold.

In some embodiments, milk products are contemplated herein that include a subset of the components (i.e., macromolecular fractions) of Tables 1A-1C. In other embodiments, milk products contemplated herein can exclude one or more of the components (i.e., macromolecular fractions) of Tables 1A-1C.

TABLE 2A
Macromolecular Content and Concentration Ranges of Functional
Nutrition Products Derived from Cow Mammary Epithelial.

| Macromolecular Fraction | Concentration, g/L |
|---|---|
| Protein¹⁻³ | |
| Total Protein | 14-60 |
| Total Casein | 8-53 |
| Total Whey Proteins | 1-24 |
| β-Casein | 3.5-18 |
| α_{S1}-Casein | 3.5-18 |
| α_{S2}-Casein | 1-6 |
| κ-Casein | 0.5-6 |
| β-Lactoglobulin | 1-8 |
| α-Lactalbumin | 0.25-3 |
| Lysozyme (μg/L) | 2.5-23 (μg/L) |
| Lactoferrin | 0.005-0.8 |
| Serum albumin | 0.03-3 |
| Lipids² | |
| Total lipids | 18-83 |
| Triacylglycerides | 15-80 |
| Diacylglycerides | 0.15-3 |
| Cholesterol | 0.1-6 |
| Phospholipids | 0.05-2 |
| Free fatty acids | 0.05-2 |
| Fatty acids² | |
| Saturated fatty acids | 8-38 |
| Myristic acid (C14:0) | 0.5-6 |
| Palmitic acid (C16:0) | 3-15 |
| Lauric acid (C12:0) | 0.3-2 |
| Monounsaturated fatty acids | 2.5-18 |
| Oleic acid (C18:1 n-9 Z) | 3-15 |
| Polyunsaturated fatty acids | |
| Linoleic acid, LA (C18:2 n-6 Z) | 0.25-3 |
| Conjugated linoleic acid, (9c,11t-CLA) | 0.025-0.23 |
| α-Linolenic acid, ALA (C18:3 n-3) | 0.25-2.3 |
| Lactose¹ | 20-90 |
| Oligosaccharides^{4, 5} | |
| Total Oligosaccharides | 0.005-0.23 |
| 6′-Sialyllactose (6′-SL) | 0.005-0.15 |
| 6′-sialyl-n-acetyllactosamine (6′-SLN) | 0.003-0.03 |
| Disialyllactose (DSL) | <0.005 |
| Galactosaminuyllactose (GNL) | 0.001-0.009 |
| 3′-Sialyllactose (3′-SL) | 0.013-0.23 |

TABLE 2B
Macromolecular Content and Concentration Ranges of Functional
Nutrition Products Derived from Cow Mammary Epithelial Cells.

| Macromolecular Fraction | Concentration, g/L |
|---|---|
| Protein¹⁻³ | |
| Total Protein | 21-50 |
| Total Casein | 12-44 |
| Total Whey Proteins | 1.5-20 |
| β-Casein | 5.3-15 |
| α_{S1}-Casein | 5.3-15 |
| α_{S2}-Casein | 1.5-5 |
| κ-Casein | 0.8-5 |
| β-Lactoglobulin | 1.5-6.3 |
| α-Lactalbumin | 0.38-2.5 |
| Lysozyme (μg/L) | 3.8-19 (μg/L) |
| Lactoferrin | 0.008-0.63 |
| Serum albumin | 0.038-2.5 |
| Lipids² | |
| Total lipids | 26-69 |
| Triacylglycerides | 23-68 |
| Diacylglycerides | 0.23-2.5 |
| Cholesterol | 0.15-5 |
| Phospholipids | 0.08-1.3 |
| Free fatty acids | 0.08-1.3 |
| Fatty acids² | |
| Saturated fatty acids | 11.3-31.3 |
| Myristic acid (C14:0) | 0.75-5 |
| Palmitic acid (C16:0) | 4.5-12.5 |
| Lauric acid (C12:0) | 0.45-1.25 |
| Monounsaturated fatty acids | 3.8-15 |
| Oleic acid (C18:1 n-9 Z) | 4.5-12.5 |
| Polyunsaturated fatty acids | |
| Linoleic acid, LA (C18:2 n-6 Z) | 0.38-2.5 |
| Conjugated linoleic acid, (9c,11t-CLA) | 0.038-0.19 |
| α-Linolenic acid, ALA (C18:3 n-3) | 0.38-1.9 |
| Lactose¹ | 30-75 |
| Oligosaccharides^{4, 5} | |
| Total Oligosaccharides | 0.0075-0.19 |
| 6′-Sialyllactose (6′-SL) | 0.0075-0.13 |
| 6′-sialyl-n-acetyllactosamine (6′-SLN) | 0.0038-0.025 |
| Disialyllactose (DSL) | <0.008 |
| Galactosaminuyllactose (GNL) | 0.0015-0.0075 |
| 3′-Sialyllactose (3′-SL) | 0.019-0.19 |

TABLE 2C
Macromolecular Content and Concentration Ranges of Functional
Nutrition Products Derived from Cow Mammary Epithelial Cells.

| Macromolecular Fraction | Concentration, g/L |
|---|---|
| Protein¹⁻³ | |
| Total Protein | 28-40 |
| Total Casein | 16-35 |
| Total Whey Proteins | 2-16 |
| β-Casein | 7-12 |
| α_{S1}-Casein | 7-12 |
| α_{S2}-Casein | 2-4 |
| κ-Casein | 1-4 |
| β-Lactoglobulin | 2-5 |
| α-Lactalbumin | 0.5-2 |
| Lysozyme (μg/L) | 5-15 (μg/L) |
| Lactoferrin | 0.01-0.5 |
| Serum albumin | 0.05-2 |
| Lipids² | |
| Total lipids | 35-55 |
| Triacylglycerides | 30-54 |
| Diacylglycerides | 0.3-2 |
| Cholesterol | 0.2-4 |
| Phospholipids | 0.1-1 |
| Free fatty acids | 0.1-1 |
| Fatty acids² | |
| Saturated fatty acids | 15-25 |
| Myristic acid (C14:0) | 1-4 |
| Palmitic acid (C16:0) | 6-10 |
| Lauric acid (C12:0) | 0.6-1 |
| Monounsaturated fatty acids | 5-12 |
| Oleic acid (C18:1 n-9 Z) | 6-10 |
| Polyunsaturated fatty acids | 0.5-10 |
| Linoleic acid, LA (C18:2 n-6 Z) | 0.5-2 |
| Conjugated linoleic acid, (9c,11t-CLA) | 0.05-0.15 |
| α-Linolenic acid, ALA (C18:3 n-3) | 0.5-1.5 |
| Lactose¹ | 40-60 |
| Oligosaccharides^{4, 5} | |
| Total Oligosaccharides | 0.01-0.15 |
| 6′-Sialyllactose (6′-SL) | 0.01-0.1 |
| 6′-sialyl-n-acetyllactosamine (6′-SLN) | 0.005-0.02 |
| Disialyllactose (DSL) | <0.01 |
| Galactosaminuyllactose (GNL) | 0.002-0.006 |
| 3′-Sialyllactose (3′-SL) | 0.025-0.15 |

TABLE 2D
Macromolecular Content and Concentration Ranges of Functional
Nutrition Products Derived from Cow Mammary Epithelial.

| Macromolecular Fraction | Concentration, g/L |
|---|---|
| Protein¹⁻³ | |
| Total Protein | 14-60 |
| Total Casein | 8-53 |
| Total Whey Proteins | 1-24 |
| β-Casein | 3.5-18 |
| α_{S1}-Casein | 3.5-18 |
| α_{S2}-Casein | 1-6 |
| κ-Casein | 0.5-6 |
| β-Lactoglobulin | 1-8 |
| α-Lactalbumin | 0.25-3 |
| Lysozyme (μg/L) | 2.5-23 (μg/L) |
| Lactoferrin | 0.005-0.8 |
| Serum albumin | 0.03-3 |
| Lipids² | |
| Total lipids | 18-83 |
| Triacylglycerides | 1.6-7 |
| Phospholipids | 9.7-45 |
| Other Lipids | 6.7-31 |
| Fatty acids² | |
| Saturated fatty acids | 8-38 |
| Myristic acid (C14:0) | 0.5-6 |
| Palmitic acid (C16:0) | 3-15 |
| Lauric acid (C12:0) | 0.3-2 |
| Monounsaturated fatty acids | 2.5-18 |
| Oleic acid (C18:1 n-9 Z) | 3-15 |
| Polyunsaturated fatty acids | |
| Linoleic acid, LA (C18:2 n-6 Z) | 0.25-3 |
| Conjugated linoleic acid, (9c,11t-CLA) | 0.025-0.23 |
| α-Linolenic acid, ALA (C18:3 n-3) | 0.25-2.3 |
| Lactose¹ | 20-90 |
| Oligosaccharides^{4, 5} | |
| Total Oligosaccharides | 0.005-0.23 |
| 6′-Sialyllactose (6′-SL) | 0.005-0.15 |
| 6′-sialyl-n-acetyllactosamine (6′-SLN) | 0.003-0.03 |
| Disialyllactose (DSL) | <0.005 |
| Galactosaminuyllactose (GNL) | 0.001-0.009 |
| 3′-Sialyllactose (3′-SL) | 0.013-0.23 |

TABLE 2E
Macromolecular Content and Concentration Ranges of Functional
Nutrition Products Derived from Cow Mammary Epithelial Cells.

| Macromolecular Fraction | Concentration, g/L |
|---|---|
| Protein¹⁻³ | |
| Total Protein | 21-50 |
| Total Casein | 12-44 |
| Total Whey Proteins | 1.5-20 |
| β-Casein | 5.3-15 |
| α_{S1}-Casein | 5.3-15 |
| α_{S2}-Casein | 1.5-5 |
| κ-Casein | 0.8-5 |
| β-Lactoglobulin | 1.5-6.3 |
| α-Lactalbumin | 0.38-2.5 |
| Lysozyme (μg/L) | 3.8-19 (μg/L) |
| Lactoferrin | 0.008-0.63 |
| Serum albumin | 0.038-2.5 |
| Lipids² | |
| Total lipids | 26-69 |
| Triacylglycerides | 2-6 |
| Phospholipids | 14-37 |
| Other lipids | 10-26 |
| Fatty acids² | |
| Saturated fatty acids | 11.3-31.3 |
| Myristic acid (C14:0) | 0.75-5 |
| Palmitic acid (C16:0) | 4.5-12.5 |
| Lauric acid (C12:0) | 0.45-1.25 |
| Monounsaturated fatty acids | 3.8-15 |
| Oleic acid (C18:1 n-9 Z) | 4.5-12.5 |
| Polyunsaturated fatty acids | |
| Linoleic acid, LA (C18:2 n-6 Z) | 0.38-2.5 |
| Conjugated linoleic acid, (9c,11t-CLA) | 0.038-0.19 |
| α-Linolenic acid, ALA (C18:3 n-3) | 0.38-1.9 |
| Lactose¹ | 30-75 |
| Oligosaccharides^{4, 5} | |
| Total Oligosaccharides | 0.0075-0.19 |
| 6′-Sialyllactose (6′-SL) | 0.0075-0.13 |
| 6′-sialyl-n-acetyllactosamine (6′-SLN) | 0.0038-0.025 |
| Disialyllactose (DSL) | <0.008 |
| Galactosaminuyllactose (GNL) | 0.0015-0.0075 |
| 3′-Sialyllactose (3′-SL) | 0.019-0.19 |

TABLE 2F
Macromolecular Content and Concentration Ranges of Functional
Nutrition Products Derived from Cow Mammary Epithelial Cells.

| Macromolecular Fraction | Concentration, g/L |
|---|---|
| Protein¹⁻³ | |
| Total Protein | 28-40 |
| Total Casein | 16-35 |
| Total Whey Proteins | 2-16 |
| β-Casein | 7-12 |
| α_{S1}-Casein | 7-12 |
| α_{S2}-Casein | 2-4 |
| κ-Casein | 1-4 |
| β-Lactoglobulin | 2-5 |
| α-Lactalbumin | 0.5-2 |
| Lysozyme (μg/L) | 5-15 (μg/L) |
| Lactoferrin | 0.01-0.5 |
| Serum albumin | 0.05-2 |
| Lipids² | |
| Total lipids | 35-55 |
| Triacylglycerides | 3-5 |
| Phospholipids | 19-30 |
| Other Lipds | 13-20 |
| Fatty acids² | |
| Saturated fatty acids | 15-25 |
| Myristic acid (C14:0) | 1-4 |
| Palmitic acid (C16:0) | 6-10 |
| Lauric acid (C12:0) | 0.6-1 |
| Monounsaturated fatty acids | 5-12 |
| Oleic acid (C18:1 n-9 Z) | 6-10 |
| Polyunsaturated fatty acids | 0.5-10 |
| Linoleic acid, LA (C18:2 n-6 Z) | 0.5-2 |
| Conjugated linoleic acid, (9c,11t-CLA) | 0.05-0.15 |
| α-Linolenic acid, ALA (C18:3 n-3) | 0.5-1.5 |
| Lactose¹ | 40-60 |
| Oligosaccharides^{4, 5} | |
| Total Oligosaccharides | 0.01-0.15 |
| 6′-Sialyllactose (6′-SL) | 0.01-0.1 |
| 6′-sialyl-n-acetyllactosamine (6′-SLN) | 0.005-0.02 |
| Disialyllactose (DSL) | <0.01 |
| Galactosaminuyllactose (GNL) | 0.002-0.006 |
| 3′-Sialyllactose (3′-SL) | 0.025-0.15 |

In some embodiments, the concentrations of components indicated in Tables 2A-2F can vary, each individually, for example, by having a concentration that is greater than that indicated by 0.1 fold, or 0.2 fold, or 0.3 fold, or 0.4 fold, or 0.5 fold, or 0.6 fold, or 0.7 fold, or 0.8 fold, or 0.9 fold, or 1, 2, 3, 4, 5, 6, 7, 8, 9, or 10 fold.

In some embodiments, the concentrations of components indicated in Tables 2A-2F can vary, each individually, for example, by having a concentration that is less than that indicated by 0.1 fold, or 0.2 fold, or 0.3 fold, or 0.4 fold, or 0.5 fold, or 0.6 fold, or 0.7 fold, or 0.8 fold, or 0.9 fold.

In some embodiments, milk products are contemplated herein that include a subset of the components (i.e., macromolecular fractions) of Tables 2A-2F. In other embodiments, milk products contemplated herein can exclude one or more of the components (i.e., macromolecular fractions) of Tables 2A-2F.

Disclosed herein, in certain embodiments, are a milk products comprising about 28-40 grams per liter (g/L) protein components, about 35-55 g/L lipid components, about 0.01-0.15 g/L milk oligosaccharides (MOs), and about 40-60 g/L lactose, wherein at least one of the protein components, lipid components, MOs, and lactose is produced by cultured cow mammary epithelial cells.

In some embodiments of the milk product, the protein component can comprise whey protein, and in some embodiments the whey protein can have a concentration of about 1-24 g/L in the milk product. In some embodiments, the protein component can comprise casein protein, and in some embodiments, casein protein can comprise one or more of beta-casein, kappa-casein, and alpha-casein. In some embodiments, the beta-casein can have a concentration of about 7-12 g/L, the kappa-casein can have a concentration of about 1-4 g/L, and the alpha-casein can have a concentration of about 9-16 g/L in the milk product. In some embodiments, the alpha-casein can comprise one or more of alpha_{S1}-casein and alpha_{S2}-casein, and in some embodiments, the alpha_{S1}-casein is at least 1.5 fold, or about 2 fold, or about 2.5 fold, or about 3 fold, or about 3.5 fold, or about 4 fold more abundant than alpha_{S2}-casein. In some embodiments, the alpha_{S1}-casein can have a concentration of about 7-12 g/L in the milk product, and in some embodiments, the alpha_{S2}-casein can have a concentration of about 2-4 g/L in the milk product. In some embodiments, the beta-casein can comprise greater than about 50% of total casein content.

In some embodiments of the milk product, the protein component can further comprise one or more of beta-lactoglobulin, alpha-lactalbumin, lysozyme, lactoferrin and serum albumin, and in some embodiments, the beta-lactoglobulin can have a concentration of about 2-5 g/L in the milk product and/or the alpha-lactalbumin can have a concentration of about 0.5-2 g/L in the milk product and/or the lysozyme can have a concentration of about 5-15 μg/L in the milk product and/or the lactoferrin can have a concentration of about 0.01-0.5 g/L in the milk product and/or the serum albumin can have a concentration of about 0.05-2 g/L in the milk product.

In some embodiments of the milk product, the lipid component can comprise one or more of triacylglycerides, diacylglycerides, saturated fatty acids, monounsaturated fatty acids, polyunsaturated fatty acids, cholesterol, and phospholipids. In some embodiments, triacylglycerides can have a concentration of about 30-54 g/L in the milk product, and in some embodiments, diacylglycerides can have a concentration of about 0.33-2 g/L in the milk product. In some embodiments, saturated fatty acids can have a concentration of about 15-25 g/L in the milk product, and in some embodiments, the saturated fatty acid component can comprise one or more of myristic acid, palmitic acid, and lauric acid, where, in some embodiments, myristic acid can have a concentration of about 1-4 g/L in the milk product, and, in some embodiments, palmitic acid can have a concentration of about 6-10 g/L in the milk product, and, in some embodiments, lauric acid can have a concentration of about 0.6-1 g/L in the milk product.

In some embodiments of the milk product, monounsaturated fatty acids can have a concentration of about 5-12 g/L in the milk product, and, in some embodiments, monounsaturated fatty acid can comprise oleic acid, which oleic acid, in some embodiments, can have a concentration of about 6-10 g/L in the milk product.

In some embodiments of the milk product, polyunsaturated fats can have a concentration of about 0.5-10 g/L in the milk product, and, in some embodiments, the polyunsaturated fats can comprise one or more of linoleic acid, conjugated linoleic acid, and alpha-linoleic acid. In some embodiments, linoleic acid can have a concentration of about 0.5-2 g/L in the milk product, and in some embodiments, conjugated linolenic acid can have a concentration of about 0.05-0.15 g/L in the milk product, and in some embodiments, alpha-linoleic acid can have a concentration of about 0.5-1.5 g/L in the milk product.

In some embodiments, linoleic acid has a concentration of about 0.5-2 g/L, conjugated linolenic acid has a concentration of about 0.05-0.15 g/L, and alpha-linoleic acid has a concentration of about 0.5-1.5 g/L in the milk product.

In some embodiments, the milk product can comprise cholesterol, which, in some embodiments can have a concentration of about 0.2-4 g/L in the milk product.

In some embodiments, the milk product can comprise phospholipids, which, in some embodiments, can have a concentration of about 0.1-1 g/L in the milk product. In some embodiments, the milk product comprises phospholipids at a concentration of at least about 5, 10, 15, 20, 25, 30, 35, 40, 45, 50, 55, 60, 65, 70, or more than 70 g/L. In some embodiments, the milk product comprises phospholipids at a concentration of about 5 to about 70, about 5 to about 65, about 5 to about 60, about 5 to about 55, about 5 to about 50, about 5 to about 45, about 5 to about 40, about 5 to about 35, about 5 to about 30, about 5 to about 25, about 5 to about 20, about 5 to about 15, about 10 to about 70, about 10 to about 65, about 10 to about 60, about 10 to about 55, about 10 to about 50, about 10 to about 45, about 10 to about 40, about 10 to about 35, about 10 to about 30, about 10 to about 25, about 10 to about 20, about 10 to about 15, about 20 to about 70, about 20 to about 65, about 20 to about 60, about 20 to about 55, about 20 to about 50, about 20 to about 45, about 20 to about 40, about 20 to about 35, about 20 to about 30, about 20 to about 25, about 30 to about 70, about 30 to about 65, about 30 to about 60, about 30 to about 55, about 30 to about 50, about 30 to about 45, about 30 to about 40, about 30 to about 35, about 40 to about 70, about 40 to about 65, about 40 to about 60, about 40 to about 55, about 40 to about 50, or about 40 to about 45 g/L. In some embodiments, the phospholipids comprise phosphatidylcholine (PC), phosphatidylethanolamine (PE), phosphatidylserine (PS), phosphatidylinositol (PI), sphingomyelin (SM), or combinations thereof.

In some embodiments, the milk product comprises phospholipids that are at least about 10%, 15%, 20%, 25%, 30%, 35%, 40%, 45%, 50%, 55%, 60%, 65%, 70%, 75%, 80%, 85%, 90%, 95%, or more than 95% of the total lipid composition. In some embodiments, the milk product comprises phospholipids that are a majority of the lipid composition. In some embodiments, the milk product comprises phospholipids that are at least about 51%, 55%, 60%, 65%, 70%, 75%, 80%, 85%, 90%, 95%, or more than 95% of the total lipid composition. In some embodiments, the phospholipids comprise phosphatidylcholine (PC), phosphatidylethanolamine (PE), phosphatidylserine (PS), phosphatidylinositol (PI), sphingomyelin (SM), or combinations thereof.

In some embodiments of the milk product, the milk oligosaccharide component can comprise one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL).

In some embodiments, the one or more milk oligosaccharides comprises 6′-Sialyllactose (6′-SL), which, in some embodiments, can have a concentration of about 0.01-0.1 g/L in the milk product.

In some embodiments, the one or more oligosaccharides comprises 6′-sialyl-n-acetyllactosamine (6′-SLN), which, in some embodiments, can have a concentration of about 0.005-0.02 g/L in the milk product. In some embodiments, the one or more oligosaccharides comprises Disialyllactose (DSL), which, in some embodiments, can have a concentration of less than about 0.01 g/L in the milk product. In some embodiments, the one or more oligosaccharides comprises Galactosaminuyllactose (GNL), which, in some embodiments, can have a concentration of about 0.002-0.006 g/L in the milk product. In some embodiments, the one or more oligosaccharides comprises 3′-Sialyllactose (3′-SL), which, in some embodiments, can have a concentration of about 0.025-0.15 g/L in the milk product. In some embodiments, the milk product can comprise about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL), and about 0.025-0.15 g/L 3′-SL (3′-sialyllactose).

In another aspect of the disclosure, the milk product comprises about 28-40 grams per liter (g/L) protein components, about 35-55 g/L lipid components, about 0.01-0.15 g/L milk oligosaccharides (MOs), and about 40-60 g/L lactose, wherein the protein components comprise one or more of whey, beta-casein, kappa-casein, alpha_{S1}-casein, alpha_{S2}-casein, beta-lactoglobulin, alpha-lactalbumin, lysozyme, lactoferrin, and serum albumin, wherein the lipid components comprises one or more of triacylglycerides, diacylglycerides, saturated fatty acids, monounsaturated fatty acids, polyunsaturated fatty acids, cholesterol, and phospholipids, wherein the milk oligosaccharide component comprise one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL), wherein saturated fatty acids comprise one or more of myristic acid, palmitic acid, and lauric acid, wherein monounsaturated fatty acids comprise oleic acid, wherein polyunsaturated fatty acids comprise one or more of linoleic acid, conjugated linoleic acid, and alpha-linoleic acid, and wherein at least one of the protein components, lipid components, MOs, and lactose is produced by cultured cow mammary epithelial cells.

In some embodiments, the milk product comprises about 2-16 g/L whey, about 7-12 g/L beta-casein, about 1-4 g/L kappa-casein, about 7-12 g/L alpha_{S1}-casein, about 2-4 g/L alpha_{S2}-casein, about 2-5 g/L beta-lactoglobulin, about 0.5-2 g/L alpha-lactalbumin, about 5-15 μg/L lysozyme, about 0.01-0.5 g/L lactoferrin, about 0.05-2 g/L serum albumin, about 30-54 g/L triacylglycerides, about 0.3-2 g/L diacylglycerides, about 15-25 g/L saturated fatty acids, about 5-12 g/L monounsaturated fatty acids, about 0.5-10 g/L polyunsaturated fatty acids, about 0.2-4 g/L cholesterol, about 0.1-1 g/L phospholipids, about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL) and about 0.025-0.15 g/L 3′-Sialyllactose (3′-SL), wherein the saturated fatty acids comprise about 1-4 g/L myristic acid, about 6-10 g/L palmitic acid, and about 0.6-1 g/L lauric acid, wherein the monounsaturated fatty acids comprises about 6-10 g/L oleic acid, and wherein the polyunsaturated fatty acids comprise about 0.5-2 g/L linoleic acid, about 0.05-0.15 g/L conjugated linoleic acid, and about 0.5-1.5 g/L alpha-linoleic acid.

In some embodiments, the milk product comprises at least about 80% of the overall macromolecular composition of cow milk. In some embodiments, the milk product comprises at least about 85% of the overall macromolecular composition of cow milk. In some embodiments, the milk product comprises at least about 90% of the overall macromolecular composition of cow milk. In some embodiments, the milk product comprises at least about 95% of the overall macromolecular composition of cow milk. In some embodiments, the milk product comprises at least about 97% of the overall macromolecular composition of cow milk. In some embodiments, the milk product comprises at least about 98% of the overall macromolecular composition of cow milk. In some embodiments, the milk product comprises at least about 99% of the overall macromolecular composition of cow milk. In some embodiments, non-protein nitrogen content comprises at least about 10% of total nitrogen content. In some embodiments, non-protein nitrogen content comprises at least about 15% of total nitrogen content. In some embodiments, non-protein nitrogen content comprises at least about 20% of total nitrogen content. In some embodiments, non-protein nitrogen content comprises at least about 25% of total nitrogen content. In some embodiments, non-protein nitrogen content comprises at least about 30% of total nitrogen content.

In some embodiments, the cow milk product does not comprise beta-lactoglobulin.

### Uses

### Nutrition Uses

Disclosed herein, in certain embodiments, are methods of providing nutrition to a human subject in need thereof, comprising administering to the subject a non-human milk product described herein (for example, a cow milk product, a bison mil product, a buffalo milk product, a yak milk product, a goat milk product, a sheep milk product, a camel milk product or a reindeer milk product). In some embodiments, the subject is a human infant. In some embodiments, the human subject is malnourished. In some embodiments, the human subject has malabsorption syndrome. In some embodiments, the human subject has wasting syndrome. In some embodiments, the human subject is geriatric.

Disclosed herein, in certain embodiments, are methods of providing nutrition to a non-human subject in need thereof, comprising administering to the subject a non-human milk product described herein. In some embodiments, the subject is a cow and the milk product is a cow milk product. In some embodiments, the subject is a bison and the milk product is a bison milk product. In some embodiments, the subject is a buffalo and the milk product is a buffalo milk product. In some embodiments, the subject is a yak and the milk product is a yak milk product. In some embodiments, the subject is a goat and the milk product is a goat milk product. In some embodiments, the subject is a sheep and the milk product is a sheep milk product. In some embodiments, the subject is a camel and the milk product is a camel milk product. In some embodiments, the subject is a reindeer and the milk product is a reindeer milk product. In some embodiments, the subject is a pig and the milk product is a pig milk product. In some embodiments, the subject is a dog and the milk product is a dog milk product. In some embodiments, the subject is a cat and the milk product is a cat milk product. In some embodiments, the subject is a horse and the milk product is a horse milk product.

### Immunotherapeutic Uses

Secretory IgA protects the intestinal epithelium from enteric toxins and pathogenic microorganisms. Through a process known as immune exclusion, sIgA promotes the clearance of antigens and pathogenic microorganisms from the intestinal lumen by blocking their access to epithelial receptors, entrapping them in mucus, and facilitating their removal by peristaltic and mucociliary activities. In addition, sIgA directly quenches bacterial virulence factors, influences composition of the intestinal microbiota by Fab-dependent and Fab-independent mechanisms, promotes retro-transport of antigens across the intestinal epithelium to dendritic cell subsets in gut-associated lymphoid tissue, and downregulates proinflammatory responses normally associated with the uptake of highly pathogenic bacteria and potentially allergenic antigens.

In certain embodiments, the sIgA binds to an antigen of a microorganism (i.e., bacterium or virus). In certain embodiments, the sIgA binds to viral or bacterial antigens capable of causing an infectious disease in a subject. In certain embodiments, the sIgA binds to viral or bacterial antigens that cause infections of respiratory or gastrointestinal epithelium. In certain embodiments, the sIgA binds antigens from microorganisms that cause enterocolitis or sepsis in infants.

### Veterinary Uses

Disclosed herein, in certain embodiments, are methods of treating and/or preventing a microbial infection in a non-human subject in need thereof, comprising administering to the subject a non-human milk product comprising a non-human IgA or a non-human sIgA described herein, or an immunotherapeutic composition comprising a non-human IgA or a non-human sIgA described herein. In some embodiments, the non-human milk product is a cow milk product and the non-human IgA or sIgA is a cow IgA or sIgA. In some embodiments, the non-human milk product is a buffalo milk product and the non-human IgA or sIgA is a buffalo IgA or sIgA. In some embodiments, the non-human milk product is a bison milk product and the non-human IgA or sIgA is a bison IgA or sIgA. In some embodiments, the non-human milk product is a yak milk product and the non-human IgA or sIgA is a yak IgA or sIgA. In some embodiments, the non-human milk product is a goat milk product and the non-human IgA or sIgA is a goat IgA or sIgA. In some embodiments, the non-human milk product is a sheep milk product and the non-human IgA or sIgA is a sheep IgA or sIgA. In some embodiments, the non-human milk product is a camel milk product and the non-human IgA or sIgA is a camel IgA or sIgA. In some embodiments, the non-human milk product is a reindeer milk product and the non-human IgA or sIgA is a reindeer IgA or sIgA. In some embodiments, the non-human milk product is a pig milk product and the non-human IgA or sIgA is a pig IgA or sIgA. In some embodiments, the non-human milk product is a dog milk product and the non-human IgA or sIgA is a dog IgA or sIgA. In some embodiments, the non-human milk product is a cat milk product and the non-human IgA or sIgA is a cat IgA or sIgA. In some embodiments, the non-human milk product is a horse milk product and the non-human IgA or sIgA is a horse IgA or sIgA.

In some embodiments, the microbial infection is a bacterial infection, fungal infection or parasitic infection. In some embodiments, the microbial infection results in an illness in the non-human animal. In some embodiments, the non-human animal is a carrier of the microbial infection, which microbial infection can result in illness in a human (e.g., C. difficile).

In certain embodiments, the microbial infection is a bacterial infection. Non-limiting examples of bacterial infections that can be treated and/or prevented include infections caused by: Streptococcus pneumoniae, Streptococcus equi, Moraxella catarrhalis, Staphylococcus aureus, Streptococcus pyogenes, Salmonella, Shigella, Campylobacter, Staphylococcus aureus, Helicobacter pylori, E. coli, C. difficile, C. perfringens type A, C. perfringens type B, C. perfringens type C, C. perfringens type D, Clostridium piliforme, Aspergillus fumigatus, Yersinia pseudotuberculosis, Y. enterocolitica, Mycobacterium avium paratuberculosis, Bordetella bronchiseptica, Neorickettsia risticii.

In certain embodiments, the microbial infection is a viral infection. Non-limiting examples of viral infection that can be treated and/or prevented include infections caused by: influenza virus (e.g., canine influenza virus, or equine influenza virus), parainfluenza virus (e.g., parainfluenza virus type 3 (PIV3)), respiratory syncytial virus, rhinovirus, coronavirus, rotavirus, torovirus, orthobunyavirus, parvovirus, cryptosporidia, herpesviruses (e.g., EHV-1, EHV-2, EHV-4 and EHV-5), equine rhinitis-A virus (ERAV), equine rhinitis-B virus (ERBV), equine adenovirus 1 (EAdV-1), equine arteritis virus (EAV), reovirus 3.

In some embodiments, the microbial infection is a parasitic infection. In some embodiments, the parasitic infection is an infection of Giardia lamblia, Entamoeba histolytica, Cryptosporidium spp., or Cystoisospora belli.

### Human Uses

Disclosed herein, in certain embodiments, are methods of treating and/or preventing a microbial infection in a human subject in need thereof, comprising administering to the subject a non-human milk product comprising human IgA or human sIgA described herein.

In some embodiments, the human subject is immuno-compromised. In some embodiments, the human subject has a disease selected from: severe combined immunodeficiency (SCID), HIV/AIDS, a cancer, or an autoimmune disease. In some embodiments, the human subject has lupus or diabetes (for example, Type I diabetes or Type II diabetes). In some embodiments, the human subject is an organ or bone marrow transplant recipient. In some embodiments, the human subject has cystic fibrosis, COPD, or non-CF bronchiectasis.

In certain embodiments, the immunotherapeutic compositions and non-human milk products comprising human IgA and/or human sIgA are administered to a human subject in an effective amount for the treatment or prevention of a microbial infection. In some embodiments, the microbial infection is a bacterial infection, fungal infection or parasitic infection.

In certain embodiments, the microbial infection is a bacterial infection. Non-limiting examples of bacterial infections that can be treated and/or prevented include infections caused by: Streptococcus pneumoniae, Moraxella catarrhalis, Staphylococcus aureus, Streptococcus pyogenes, Salmonella, Shigella, Campylobacter, Staphylococcus aureus, Helicobacter pylori, E. coli, C. difficile, or Vibrio cholerae.

In certain embodiments, the microbial infection is a viral infection. Non-limiting examples of viral infection that can be treated and/or prevented include infections caused by: influenza virus, parainfluenza virus, respiratory syncytial virus, rhinovirus, and human metapneumovirus.

In some embodiments, the microbial infection is a parasitic infection. In some embodiments, the parasitic infection is an infection of Giardia lamblia, Entamoeba histolytica, Cryptosporidium spp., or Cystoisospora belli.

In some embodiments, the microbial infection is a fungal infection. In some embodiments, the fungal infection is an infection of Aspergillus, Cryptococcus, or Pneumocystis jirovecii.

In some embodiments, the infection is a gastrointestinal infection. In some embodiments, the infection is bacterial or viral gastroenteritis. In some embodiments, the infection is a Helicobacter pylori infection.

In some embodiments, the microbial infection is Candidiasis.

In some embodiments, the microbial infection is a respiratory infection. In some embodiments, the respiratory infection is pneumonia, bronchitis, Aspergillosis, or Cryptococcosis. In some embodiments, the respiratory infection is an infection by B. cepacia, P. aeruginosa S. aureus, Aspergillus, Cryptococcus, or Pneumocystis.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Production of Cultured Milk Components from Cow Mammary Epithelium

In this example, cow mammary epithelium is recapitulated and milk product is produced in vitro. The system and process described is exemplary and can be scaled to produce multiliter volumes of milk components. Cow mammary epithelial cells are expected to form a polarized monolayer on bioreactor fibers that have been precoated with one or more of laminin and collagen or other extracellular matrix proteins, as well as on uncoated fibers. When confluent, the monolayer forms a barrier that divides the intra- and extracapillary space (ECS), with the basal surface attached to the fibers and the apical surface oriented toward the ECS. Cultured milk component production is stimulated by addition of prolactin to the media. The secreted milk components are collected from the ECS and submitted for downstream analyses of the protein, lipid, and carbohydrate content in comparison to cow milk produced in vivo.

Materials for use in this Example are shown in Table 3.

TABLE 3
Materials

| | | Catalog (or other |
|---|---|---|
| Item | Supplier | Identification) Number |
| Primary cow mammary epithelial | Academic community | MAC-T ¹ |
| cells | resource | |
| Dulbecco's Modified Eagle's | Sigma | D5030 |
| Medium (DMEM) | | |
| Fetal cow serum | Sigma | F4135 |
| Insulin | Sigma | I6634 |
| Hydrocortisone | Sigma | H0888 |
| Dulbecco's phosphate buffered | ATCC | ATCC 30-2200 |
| saline (D-PBS) | | |
| Trypsin-EDTA | ATCC | PCS-999-003 |
| Trypsin Neutralizing Solution | ATCC | PCS-999-004 |
| EHS (Laminin-1/111) | Sigma | L2020-1MG |
| Collagen-IV | Sigma | C5533-5MG |
| Prolactin | Shenandoah Biotechnology | 100-45-500ug |

¹ Huynh, HT, et al., “Establishment of cow mammary epithelial cells (MAC-T): an in vitro model for cow lactation”, Exp Cell Res. 1991 December; 197(2): 191-9.

### Procedures

### Expansion of Primary Cow Mammary Epithelial Cells (BMECs)

Cow mammary epithelial cells (1 ampoule; 5×10⁵ cells) are expanded into one collagen-IV-coated T300 flask (or two T175 flasks) in DMEM supplemented with fetal cow serum, insulin, and hydrocortisone, as listed in Table 3. Once an appropriate cell number is obtained, cells are rinsed with D-PBS and collected from the plates using trypsin-EDTA. Once cells are detached, trypsin activity is halted using Trypsin Neutralizing Solution. Cells are resuspended in medium and seeded into a hollow fiber bioreactor (Fibercell Systems), prepared as described below.

### Preparation of Hollow Fiber Bioreactor (C2025D, 20 kD MWCO)

Prior to seeding, a bioreactor cartridge (Fibercell Systems) is prepared by pre-culturing with PBS for a minimum of 24 hours. The bioreactor cartridge is optionally pre-coated by adding about 50-100 μg of one or more of collagen I, collagen IV, laminin-111 (e.g., laminin-111 isolated from Engelbreth-Holm Swarm tumor), alpha-4, alpha-5, fibronectin, and/or entactin in 3.2 mL of PBS and allowing ultrafiltration across the fiber at room temperature overnight. The uncoated or precoated cartridge is exchanged with medium and incubated overnight at room temperature. The medium is then exchanged with the cells collected from the T300 (or T175) flask(s). The reservoir volume is no more than 125 mL. The cartridge is rotated 180 degrees after seeding the cells.

### Cell Growth in the Bioreactor and Prolactin Stimulation

After seeding the bioreactor, cells are grown in DMEM supplemented with fetal cow serum, insulin, and hydrocortisone.

Before stimulation of milk secretion, the medium in the ECS is flushed and replaced with PBS. To stimulate milk component secretion, lactogenic medium (medium supplemented with 5 μg/mL prolactin) is added. The lactogenic medium can also be supplemented with an elevated concentration of glucose and the essential dietary precursors for milk fatty acids, linoleic acid and α-linolenic acid. The bioreactor is maintained for 10 days with sampling as described below.

### Harvesting and Sample Preparation

Samples, comprised of supernatant from the ECS and an equivalent volume of media from the reservoir, are collected once daily for 10 days after addition of prolactin to the media. The samples are spun in a centrifuge to collect any debris and resuspended in an equivalent volume of PBS. The supernatants from the ECS and media samples are divided into 0.5 mL aliquots in microfuge tubes and frozen at −80° C. The pellet debris is resuspended in a volume of PBS equivalent to the original sample and frozen at −80° C. Samples are processed to determine relative concentrations of milk components produced.

### Scaling for Multiliter Production

To scale for multiliter production, the preceding procedure with relative adjustments in reagent volumes for a larger bioreactor (e.g., Fibercell Systems cat. no. C2018) is performed.

### Example 2: Lipid Profiles of Bovine Cell Cultured Milk Products

Samples of cell cultured milk products produced by a bovine mammary epithelial cell (MEC) line (MAC-T) were submitted for a lipidomic analysis (UNC Mass Spectrometry Core). Presented here are the observed lipid profiles of cell cultured bovine milk products compared with naturally produced bovine milk. The lipid compositions of these cell cultured milk product examples demonstrate a higher proportion of phospholipids relative to naturally produced milk. Within the phospholipid class, the proportions of the major constituents, including phosphatidylcholine (PC), phosphatidylethanolamine (PE), phosphatidylserine (PS), phosphatidylinositol (PI), and sphingomyelin (SM), are similar between naturally occurring and cell cultured bovine milk. Milk phospholipids are associated with beneficial effects for growth and development and represent an important class of dietary components that have applications in a variety of human health and veterinary product categories.

### Methods

### Naturally Produced and Cell Cultured Milk Samples

Raw bovine milk was obtained from a local dairy farm in the Raleigh-Durham, NC area. The sample was transported to the laboratory on ice and frozen at −80° C. until the time of analysis.

Cell cultured bovine milk product examples were obtained from a bioprocess in which the bovine MEC cell line MAC-T was inoculated into the extracapillary compartment of a hollow fiber bioreactor and subsequently cultured in DMEM/F12 supplemented with 10% FBS. Glucose utilization was monitored, and media was changed when glucose levels were depleted by 50%. After 14 days of culture, recombinant prolactin was dosed into the culture media (400 ng/mL), and the cell cultured milk product was harvested from the extracapillary compartment during each media change during a subsequent 4 weeks of culture. Product samples were centrifuged at 300×g for 15 min to separate insoluble material from the aqueous sample. The pellets were resuspended in an equivalent volume of PBS. All samples were stored at −80° C. until the time of analysis.

LIPIDOMIC analysis

Samples of naturally produced and cell cultured bovine milk were submitted for lipidomic analysis at the UNC Mass Spectrometry Core.

To prepare each sample for analysis, 500 μL was extracted using a liquid-liquid partition with methanol (300 μL) and MTBE (1 mL). The upper layer was used for lipidomics. Avanti's deuterated lipid mix, Equisplash, was used as an internal standard. This was spiked into the methanol at 1.5 μg/mL and used for extraction. The extracts were centrifuged at 20,000 rcf for 10 minutes. The top layer was removed, dried down, and reconstituted in 150 μL of IPA for analysis.

Analysis was performed using a Thermo Q Exactive Plus coupled to a Waters Acquity H-Class LC. A 100 mm×2.1 mm, 2.1 μm Waters BEH C18 column was used for separations. The following mobile phases were used: A—60/40 ACN/H20 B—90/10 IPA/ACN; both mobile phases had 10 mM Ammonium Formate and 0.1% Formic Acid. A flow rate of 0.2 mL/minute was used. Starting composition was 32% B, which increased to 40% B at 1 minute (held until 1.5 min) then 45% B at 4 minutes. This was increased to 50% B at 5 minutes, 60% B at 8 minutes, 70% B at 11 minutes, and 80% B at 14 minutes (held until 16 minutes). At 16 minutes the composition switched back to starting conditions (32% B) and was held for 4 minutes to re-equilibrate the column. Samples were analyzed in positive/negative switching ionization mode with top 5 data-dependent fragmentation.

Raw data was analyzed by LipidSearch. Lipids were identified by MS2 fragmentation (mass error of precursor=5 ppm, mass error of product=8 ppm). The identifications were generated individually for each sample and then aligned.

### Data Analysis

Lipid profiles generated from the analysis include the determination of the relative proportions of triglyceride (TG), phospholipid, and “other” lipid classes. Additionally, phospholipid profiles were generated by determining the relative proportions of PC, PE, PS, PI, and SM within the total phospholipid class. Data presented for Product Example 1 and Product Example 2 represent the combined profiles for the soluble and insoluble portions of the product harvested from the extracapillary compartment of a hollow fiber bioreactor, assessed across multiple intervals of the production phase, where each interval corresponds to the consumption of a single bottle of culture media.

### Results

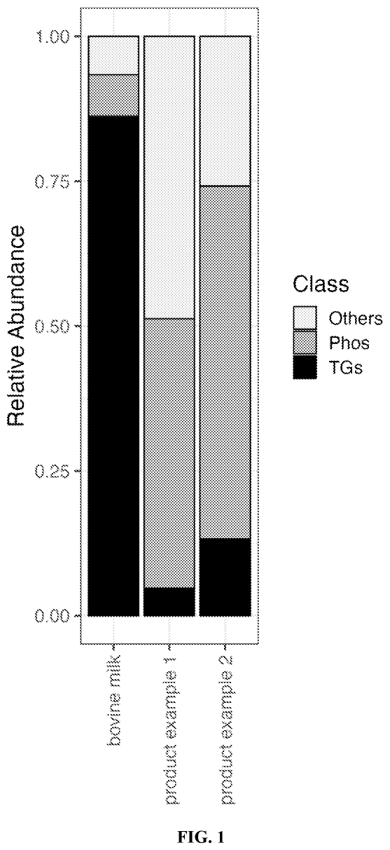

Naturally occurring and cell cultured bovine milk samples differed with respect to the relative content of TG, phospholipid, and other lipid classes. Whereas TG represented the predominant lipid class in naturally produced bovine milk, phospholipids represented a much higher proportion of the lipids present in cell cultured bovine milk (Table 4 and FIG. 1).

TABLE 4
Proportion of triglycerides, phospholipids, and other lipid
classes relative to total lipid in naturally produced
bovine milk and cell cultured bovine milk products

| | Bovine | Product | Product |
|---|---|---|---|
| | Milk | Example 1 | Example 2 |
| TG | 86.2% | 4.7% | 13.3% |
| PL | 7.2% | 46.5% | 60.9% |
| Other | 6.6% | 48.7% | 25.8% |

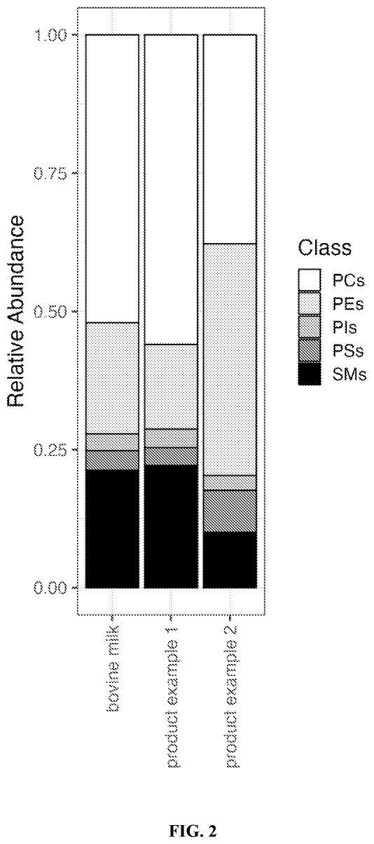

A more detailed analysis of the subclasses of phospholipids indicates that the relative proportions of PC, PE, PI, PS, and SM are similar between cell cultured bovine milk and naturally produced bovine milk (Table 5 and FIG. 2).

TABLE 5
Proportion of phospholipid subclasses relative
to total phospholipids in naturally produced bovine
milk and cell cultured bovine milk products

| | Bovine | Product | Product |
|---|---|---|---|
| | Milk | Example 1 | Example 2 |
| PE | 20.1% | 15.3% | 41.9% |
| PC | 52.0% | 56.0% | 37.8% |
| PI | 3.1% | 3.4% | 2.7% |
| PS | 3.5% | 3.2% | 7.6% |
| SM | 21.3% | 22.1% | 10.0% |

### CONCLUSION

The phospholipids in milk are derived from the MEC plasma membrane, which envelops structures such as the milk fat globule and milk extracellular vesicles as they are secreted during milk biosynthesis. (Anto L, Warykas S W, Torres-Gonzalez M, Blesso C N. Milk Polar Lipids: Underappreciated Lipids with Emerging Health Benefits. Nutrients. 2020; 12(4).) These polar lipids represent a small proportion of total milk fat but are increasingly recognized for their beneficial effects in gut, brain, heart, and immune function.

Among the polar lipids present in milk, SM is the most potently bioactive and has been studied the most extensively for its health-promoting effects. (Id.) Dietary SM has been shown to have anti-inflammatory effects in the gut in rodent models and milk SM specifically has been shown to improve lipid metabolism, gut dysbiosis, and inflammation. (Mazzei J C, Zhou H, Brayfield B P, Hontecillas R, Bassaganya-Riera J, Schmelz E M. Suppression of intestinal inflammation and inflammation-driven colon cancer in mice by dietary sphingomyelin: importance of peroxisome proliferator-activated receptor gamma expression. J Nutr Biochem. 2011; 22(12):1160-1171 and Norris G H, Milard M, Michalski M C, Blesso C N. Protective properties of milk sphingomyelin against dysfunctional lipid metabolism, gut dysbiosis, and inflammation. J Nutr Biochem. 2019; 73:108224). In the only reported human trial, dietary supplementation with milk polar lipids improved cardiovascular risk factors among overweight post-menopausal women. (Vors C, Joumard-Cubizolles L, Lecomte M, et al. Milk polar lipids reduce lipid cardiovascular risk factors in overweight postmenopausal women: towards a gut sphingomyelin-cholesterol interplay. Gut. 2020; 69(3):487-501).

This Example shows milk products described herein have a higher proportion of phospholipids relative to naturally produced milk.

The foregoing examples are illustrative of the present disclosure and are not to be construed as limiting thereof. Although the disclosure has been described in detail with reference to preferred embodiments, variations and modifications exist within the scope and spirit of the disclosure as described and defined in the following claims.

What is claimed is:

1. A cow milk product, comprising:
a. about 28-40 grams per liter (g/L) protein components;
b. about 35-55 g/L lipid components;
c. about 0.01-0.15 g/L milk oligosaccharides (MOs); and
d. about 40-60 g/L lactose,
wherein the protein components, lipid components, MOs, and lactose are produced by cultured cow mammary epithelial cells.

2. The milk product according to claim 1, wherein the protein component comprises whey protein.

3. The milk product according to claim 2, wherein the whey protein has a concentration of about 1-24 g/L in the milk product.

4. The milk product according to either of claim 2 or 3, wherein the protein component further comprises casein protein.

5. The milk product according to claim 1, wherein the protein component comprises casein protein.

6. The milk product according to any one of claim 1, 4, or 5, wherein the protein component comprises beta-casein, kappa-casein and alpha-casein.

7. The milk product according to claim 6, wherein the beta-casein has a concentration of about 7-12 g/L, the kappa-casein has a concentration of about 1-4 g/L, and the alpha-casein has a concentration of about 9-16 g/L in the milk product.

8. The milk product according to any one of claims 5-7, wherein the alpha-casein comprises one or more of alpha_{S1}-casein and alpha_{S2}-casein.

9. The milk product according to claim 8, wherein the alpha_{S1}-casein is more than about 3-fold more abundant than alpha_{S2}-casein.

10. The milk product according to claim 9, wherein the alpha_{S1}-casein has a concentration of about 7-12 g/L in the milk product.

11. The milk product according to either of claim 9 or 10, wherein the alpha_{S2}-casein has a concentration of about 2-4 g/L in the milk product.

12. The milk product according to any one of claims 6-11, wherein the beta-casein comprises greater than about 50% of total casein content.

13. The milk product according to any one of claims 2-12, wherein the protein component further comprises one or more of beta-lactoglobulin, alpha-lactalbumin, lysozyme, lactoferrin and serum albumin.

14. The milk product according to claim 13, wherein the beta-lactoglobulin has a concentration of about 2-5 g/L in the milk product.

15. The milk product according to one of claims 1-11, wherein the milk product does not compromise beta-lactoglobulin.

16. The milk product according to claim 13, wherein the alpha-lactalbumin has a concentration of about 0.5-2 g/L in the milk product.

17. The milk product according to claim 13, wherein the lysozyme has a concentration of about 5-15 μg/L in the milk product.

18. The milk product according to claim 13, wherein the lactoferrin has a concentration of about 0.01-0.5 g/L in the milk product.

19. The milk product according to claim 13, wherein the serum albumin has a concentration of about 0.05-2 g/L in the milk product.

20. The milk product according to any of claims 1-19, wherein the lipid component comprises one or more of triacylglycerides, diacylglycerides, saturated fatty acids, monounsaturated fatty acids, polyunsaturated fatty acids, cholesterol, and phospholipids.

21. The milk product according to claim 20, wherein the triacylglycerides have a concentration of about 30-54 g/L in the milk product.

22. The milk product according to either claim 20 or 21, wherein the diacylglycerides have a concentration of about 0.33-2 g/L in the milk product.

23. The milk product according to any one of claims 20-22, wherein the saturated fatty acids have a concentration of about 15-25 g/L in the milk product.

24. The milk product according to any one of claims 20-23, wherein the saturated fatty acids comprise one or more of myristic acid, palmitic acid, and lauric acid.

25. The milk product according to claim 24, wherein myristic acid has a concentration of about 1-4 g/L in the milk product.

26. The milk product according to either of claim 24 or 25, wherein palmitic acid has a concentration of about 6-10 g/L in the milk product.

27. The milk product according to any one of claims 24-26, wherein lauric acid has a concentration of about 0.6-1 g/L in the milk product.

28. The milk product according to any one of claims 20-27, wherein monounsaturated fatty acids have a concentration of about 5-12 g/L in the milk product.

29. The milk product according to claim 28, wherein the monounsaturated fatty acid comprises oleic acid.

30. The milk product according to claim 29, wherein the oleic acid has a concentration of about 6-10 g/L in the milk product.

31. The milk product according to any one of claims 20-30, wherein polyunsaturated fats have a concentration of about 0.5-10 g/L in the milk product.

32. The milk product according to claim 31, wherein the polyunsaturated fats comprise one or more of linoleic acid, conjugated linoleic acid, and alpha-linoleic acid.

33. The milk product according to claim 32, wherein linoleic acid has a concentration of about 0.5-2 g/L in the milk product.

34. The milk product according to either of claim 32 or 33, wherein conjugated linolenic acid has a concentration of about 0.05-0.15 g/L in the milk product.

35. The milk product according to any one of claims 32-34, wherein alpha-linoleic acid has a concentration of about 0.5-1.5 g/L in the milk product.

36. The milk product according to either of claim 31 or 32, wherein linoleic acid has a concentration of about 0.5-2 g/L, conjugated linolenic acid has a concentration of about 0.05-0.15 g/L, and alpha-linoleic acid has a concentration of about 0.5-1.5 g/L in the milk product.

37. The milk product according to any one of claims 20-36, wherein cholesterol has a concentration of about 0.2-4 g/L in milk product.

38. The milk product according to any one of claims 20-37, wherein the phospholipids have a concentration of about 0.1-1 g/L in the milk product.

39. The milk product according to any one of claims 20-37, wherein the phospholipids have a concentration of about 10-45 g/L in the milk product.

40. The milk product according to any one of claims 1-39, wherein the milk oligosaccharide component comprises one or more of 6′-Sialyllactose (6′-SL), 6′-sialyl-n-acetyllactosamine (6′-SLN), Disialyllactose (DSL), Galactosaminuyllactose (GNL) and 3′-Sialyllactose (3′-SL).

41. The milk product according to claim 49, wherein the one or more milk oligosaccharides comprises 6′-Sialyllactose (6′-SL), which oligosaccharide has a concentration of about 0.01-0.1 g/L in the milk product.

42. The milk product according to either claim 40 or 41, wherein the one or more oligosaccharides comprises 6′-sialyl-n-acetyllactosamine (6′-SLN), which oligosaccharide has a concentration of about 0.005-0.02 g/L in the milk product.

43. The milk product according to any one of claims 40-42, wherein the one or more oligosaccharides comprises Disialyllactose (DSL), which oligosaccharide has a concentration of less than about 0.01 g/L in the milk product.

44. The milk product according to any one of claims 40-43, wherein the one or more oligosaccharides comprises Galactosaminuyllactose (GNL), which oligosaccharide has a concentration of about 0.002-0.006 g/L in the milk product.

45. The milk product according to any one of claims 40-44, wherein the one or more oligosaccharides comprises 3′-Sialyllactose (3′-SL), which oligosaccharide has a concentration of about 0.025-0.15 g/L in the milk product.

46. The milk product according to any one of claims 40-45, wherein the milk product comprises about 0.01-0.1 g/L 6′-Sialyllactose (6′-SL), about 0.005-0.02 g/L 6′-sialyl-n-acetyllactosamine (6′-SLN), less than about 0.01 g/L Disialyllactose (DSL), about 0.002-0.006 g/L Galactosaminuyllactose (GNL), and about 0.025-0.15 g/L 3′-SL (3′-sialyllactose).

47. A milk product according to any one of claims 1-46 that is sterile.

48. A milk product according to claim 47 that is sterile without pasteurization.

49. A milk product according to either of claim 47 or 48 that is free of immunoglobulin protein.

50. The milk product according to any of claims 1-49, wherein the milk product comprises at least about 80%, or at least about 85%, or at least about 90%, or at least about 95%, or at least about 97%, or at least about 98%, or at least about 99%, of the overall macromolecular composition of cow milk.

51. The milk product according to any of claims 1-50, wherein non-protein nitrogen content comprises at least about 10%, or at least about 15%, or at least about 20%, or at least about 25%, or at least about 30% of total nitrogen content.

52. The milk product according to any of claims 1-51, wherein the antioxidant capacity of the milk products is substantially different from the antioxidant capacity of cow milk.

53. The milk product according to any of claims 1-52, wherein the milk product does not comprise or is substantially free of one or more contaminants and toxins of cow milk.

54. The milk product according to any of claims 1-52, wherein the milk product does not comprise or is substantially free of one or more contaminants of cow milk selected from the group comprising hormones (e.g., pituitary, steroid, hypothalamic, and thyroid hormones), gastrointestinal peptides (e.g., nerve and epidermal growth factors, and the growth inhibitors MDGI and MAF), rBGH or recombinant cow growth hormone (a genetically engineered hormone injected into cows to increase milk production), pus from infected cow udders, and/or antibiotics or pharmaceuticals which have been administered to cows.

55. The milk product according to any of claims 1-52, wherein the milk product does not comprise or is substantially free of one or more pathogens or microorganisms of cow milk.

56. The milk product according to any of claims 1-52, wherein the milk product does not comprise or is substantially free of one or more pathogens or microorganisms of cow milk selected from the group comprising Brucella, Campylobacter jejuni, Coliforms, Coxiella burnetii, Escherichia coli, Listeria monocytogenes, Mycobacterium bovis and tuberculosis, Mycobacterium paratuberculosis, Psychrotrophic Bacteria, Salmonella spp., Yersinia enterocolitica, Proteobacteria, Bacteroidetes, Firmicutes, Actinobacteria, Acidobacteria, Pseudomonas, Brevibacteriaceae, Corynebacteriaceae, Staphylococcaceae, Arthrobacter, Cronobacter, Ruminococcus and/or Faecalibacterium.
